(19) United States
(12) Patent Application Publication
(10) Pub. No.: US 2009/0305397 A1
(43) Pub. Date: Dec. 10, 2009

(54) Cellular entity maturation and transportation systems

(76) Inventors: John Robert Dodgson, London (GB); Robin Fortt, London (GB); Malcolm Austen, Middlesex (GB); Bryan Miller, Middlesex (GB); Ray Cattini, Hampshire (GB)

Correspondence Address: Goodwin Procter LLP;Attn: Patent Administrator, 135 Commonwealth Drive, Menlo Park, CA, 94025-1105, US

(21) Appl. No.: 11/908,845

(22) PCT Filed: Mar. 16, 2006
(86) PCT No.: PCT/GB2006/000972

§ 371 Date: Jul. 28, 2009

(30) Foreign Application Priority Data
Mar. 16, 2005 (GB) 0505379.8

(51) Int. Cl. C12M 1/14

(52) U.S. Cl. 435/305.3; 435/307.1

(57) ABSTRACT

A device for transporting at least one cellular entity during culture or maturation, the device comprising a substrate having one or more wells, said one or more wells being adapted to hold a cellular entity in a fluid, lid means for preventing entry or exit of the cellular entity from the one or more wells and fluid transport means connecting the one or more wells to enable flow of fluid or diffusion of chemical species. The apparatus alternatively or in addition comprise a module for transporting a payload at a controlled temperature, the module comprising an outer housing, an outer thermally insulating region, an inner thermally insulating region, and a heat sinking region located between the inner and outer thermally insulating regions, the inner thermally insulating region defining a cavity for receiving a payload, and heating means.

This invention relates to a system and method for culturing cells, oocytes, embryos, maturing ova or other cellular structures in vitro. It also relates to means for transportation of cells, ova, embryos, oocytes or other cellular structures or entities.

Various apparatus and methods are known for maturing ova and culturing embryos in vitro. In standard practice these processes are achieved using conventional tools such as pipettes for manipulation of an ovum or embryo, and Petri dishes to contain the ovum or embryo and maturation or culture medium. The ova or embryos are usually cultured in an incubator in conditions of controlled temperature and gas environment. They may be cultured singly or in groups, and for ova in particular, may be cultured in the presence of other cells, such as cumulus cells. Maturation or culture is often done in microdrops of medium in a Petri dish, the medium covered by an inert oil, the dish having gas access to the environment in the incubator. In some conventional maturation or culture procedures the volume of the medium environment in which the ovum or embryo is contained is important there is evidence in some methods that maturation and culture is more successful if several ova or embryos are present together in a small volume of medium. This autocrine effect is thought to result from trace chemical substances produced by a first ovum or embryo affecting the development of a second. However, it is also advantageous in certain circumstances to track the identity of individual ova or embryos and conventional apparatus in general does not allow the embryos or ova to be kept separate while allowing exchange of chemical substances between them. The well-of-wells (WOW) method of Vajta et al. as disclosed in WO 0 102 539 allows this to be done, but does not close the wells against exit of the embryos and so is not suitable for use in a transportable device.

The medium is usually buffered against changes in pH; this buffer may be based on bicarbonate/CO₂, in which case the partial pressure of CO₂ in the external gaseous environment is important, and it may be based in whole or part on other buffer systems, for example HEPES, in which case the gaseous environment may be less closely controlled or in some circumstances not controlled at all. The medium may be of nominally constant composition during maturation or culture, or may be changed, for renewed media of the same nominal composition, or a new medium to modify the medium conditions in order for example to assist or control the process of maturation or culture. In particular, in certain methods for culture of embryos it is known to be advantageous to culture the embryos initially in serum-free medium, changing to medium containing serum (often fetal calf serum, FCS) later in culture. In the case of maturation of ova, it is known that the progress of maturation may be controlled by addition of species to the maturation medium or their removal from it by replacing the medium with fresh medium. This may be particularly advantageous if the ova or embryos are to be transported during the maturation or culturing process, for example from a location at which the ova are harvested or the embryo created, and a second location where the ova might be used or the embryo implanted. Conventionally medium is changed by moving the ovum or embryo by pipetting from one medium to another, for example from one microdrop to another in a common culture dish. This uses simple apparatus but suffers from several disadvantages: the ova and embryos are delicate and can be damaged by pipetting; an amount of medium is necessarily transferred from one medium environment to another, which is significant especially in the small volume of a microdrop, and gives the possibility that substances from the old medium active at very low concentrations may be transferred into the new medium, unless sequential washing steps are used; the transfer process is slow and requires skilled personnel; and the transfer cannot be done remotely, so cannot be done in transit or outside a fully equipped laboratory setting.

In the description that follows reference will be made to culture of embryos as an example of the function of apparatus and description of the method. Many of the processes can also be applied to maturation of ova and culturing of cells or other cellular entities and it will be apparent to those skilled in the art how this application can be made, with appropriately chosen dimensions for the different size scales of embryos, ova and cells. Therefore the terms maturation and culturing, and ova and embryos and cells, are used interchangeably in the following and where convenient referred to collectively as >objects=. Where specific features of the invention apply to maturation of ova, or to culturing of embryos, this will be noted.

A number of apparatus and methods have been proposed to alleviate these and other problems in the conventional art.

Beebe et al. U.S. Pat. No. 6,193,647, U.S. Pat. No. 6,695,765 have proposed a system of approximately embryo-sized microchannels in which the embryos reside, being located at a constriction within the microchannels by entrainment in flow along the channels, that flow causing them to roll along the channel in contact with one of the channel walls. This apparatus achieves close control of the medium environment of the embryo, but suffers from the disadvantages, among others, that it does not provide a means of positive location of the embryo against flow of the medium in the reverse direction, which tends to move the embryo away from the constriction; it does not provide ready means of gas exchange between the medium and an external gas environment, and does not provide a ready means of storage of a number of embryos in individual locations while tracking their identity—i.e. it is possible in the apparatus and method of U.S. Pat. No. 6,193,647 for the embryos to move from one retention position to another, so losing information as to their identity. No adaptation is disclosed which will make the apparatus suitable for use in transportation, in which potential problems of the embryos moving under gravity or motion will arise.

Campbell, et al. U.S. 2002 0 068 358 have proposed an apparatus for embryo culture which is adapted for transportation, in which the embryo is retained in a well which is capable of being closed in such a way that the embryo is positively retained, and which has a supply of medium and flow generating means which allows the medium in the well to be replaced under remote or automatic control. U.S. 2002 0 068 358 also discloses means to monitor and/or control parameters in the medium or the well, such as temperature, pH, and chemical constituents, though details of the apparatus showing exactly how this is to be achieved are not disclosed. The apparatus and method of U.S. 2002 0 068 358 are poorly adapted to shipping a number of embryos in a controlled chemical environment while keeping track of their identity—there is no means of segmenting embryos in a common well or wells; the well is considerably larger than the embryo, so giving poor control of the medium environment and a long time and large volume of medium for complete exchange of a first medium for a second; access to the well is down a long inlet tube or by entrainment in a microchannel and cannot readily be achieved using conventional pipettes; the design is not suitable for use with conventional microscopy.

Thompson et al., U.S. Pat. No. 6,673,008, disclose a method and apparatus for culturing of embryos in which the embryo is cultured in medium in a tank, the tank being supplied with medium from one or more reservoirs, and optionally provided with sensors for, for example, temperature, pH, dissolved O₂, ions in solution or metabolic products from the respiration of the embryo, allowing the medium around the embryo to be changed in response to conditions in the medium or to a programme stored in a control unit. The apparatus as disclosed in U.S. Pat. No. 6,673,008 comprises macro-scale devices enclosing a significant volume of solution, and the tanks of the invention are of large volume (10-50 ml), so requiring an even larger volume of medium in order to replace a first medium with a second. The device is not self-contained, in that it uses separate reservoirs and flow system components external to the apparatus and is not adapted for transportation. No means of gas (CO₂, air) perfusion of the embryos inside the tank is disclosed, except by means of flow of newly gas-enriched medium from the reservoir. In a practical transportation apparatus, the size of the apparatus and hence the volume of medium surrounding the embryo is advantageously smaller than specified in U.S. Pat. No. 6,673,008, and so a means to allow gas equilibration with the medium around the embryos is preferred.

Van den Steen et al., U.S. 2004 0 234 940, disclose a micro-chamber arrangement for development of embryos that allows flow of medium through a chamber based on a stacked array of sieve-like components that retain embryos in individual compartments. The embryos are located in the compartments and the stack of sieve-like components is then assembled to enclose them. The compartments are illustrated as being approximately embryo-sized, but the illustration in US 2004 0 234 940 is purely schematic and no means is disclosed of fabricating such a structure. No lid or other means of closure is disclosed that will allow transportation of the apparatus.

Vajta et al. WO 0 102 539 disclose a method of culturing embryos in an array of small wells located at the base of a larger well (known as the well-of-wells method). This allows embryos to be located separately in a common medium, but does not include means to retain the embryos in situ if the medium or the device comprising the well is disturbed. Consequently it is unsuitable for transport of embryos outside the laboratory environment. Also, as the method is based on an open well, it relies on exchange of gas from, and heating by, the environment in an incubator. Further, no means is disclosed of changing the composition of the medium other than by pipetting the medium into and out of the larger well.

Vajta et al. U.S. Pat. No. 6,399,375 disclose transport of ova or embryos in capillary-like straws, as used for embryo transfer, the straw having optionally sealed ends, and in which the maturation or culture process can take place during transport, but this does not allow for exchange of medium during transport.

Transport devices for embryos or ova are known, for example as manufactured by Cryologic Pty (Australia) (www.cryologic.com, www.biogenics.com) which maintain constant temperature during transport over a period of hours or days, but which can not maintain a constant gaseous environment for exchange with medium in the inner containment. The inner containment is typically in the form of vials, straws or capillaries and again there is no means for exchange of medium during transport.

A further problem with devices of the prior art disclosed in U.S. 2002 0 068 358, U.S. Pat. No. 6,673,008 and U.S. 2004 0 234 940 is that they are not adapted to be small or of low aspect ratio (such as for example straws), so requiring increased volume to contain them with consequently increased power and insulation requirements to maintain their conditions during transport. This leads to the shipping time being limited and so the contents are vulnerable to delays in shipping. Additionally, apparatus presently commercially available are insufficiently well insulated and are capable of maintaining temperature by heating, but not by cooling the sample, and so the embryos and ova are vulnerable if they encounter prolonged periods of high ambient temperature.

In the following the terms ‘cellular entity’, ‘object’ and ‘embryo’ are used interchangeably for an ovum, embryo or other cellular entity that is located within the apparatus and used in the method of the invention. Relevant parts of the apparatus can be sized according to the typical dimensions of the object to be housed. Cells other than embryos, ova and the like will be smaller, and the embodiments of the invention apply to these also given the relevant parts are sized accordingly.

According to a first aspect of the invention, there is provided a device as specified in claims 1 to 23.

According to a second aspect of the invention there is an apparatus as specified in claims 24 to 35.

According to a third aspect of the invention there is provided a transport module as specified in claim 36 to 38.

The device, apparatus and module of the present invention, among other things, allows transportation of cellular entities in a reproducible and stable environment without the need for regular operator intervention.

Mention herein with regard to the flow of fluid between wells can also relate to the diffusion of chemical species/molecules therebetween.

In one embodiment, the invention provides an apparatus for culturing or maturing of cellular entities, the apparatus comprising: a device comprising a base with one or more wells opening to a surface of the base, a lid which acts to close each well against entry or exit of a cellular entity, permeation means to allow transport of molecules to the medium in the well(s) from a gas supply within the apparatus.

According to a further embodiment, the invention provides an apparatus for culturing or maturing of cellular entities, the apparatus comprising: a device comprising a base with multiple wells opening to a surface of the base, a lid which acts to close each well against entry or exit of a cellular entity, means for chemical communication between the wells, adapted so that the cellular entities are retained in their original wells, and physical contact between cellular entities contained in adjoining wells is prevented.

According to a further embodiment, the invention provides an apparatus for culturing or maturing of cellular entities, the apparatus comprising: —a device comprising a base with one or more wells opening to a surface of the base, a lid which acts to close each well against entry or exit of a cellular entity and means to modify the composition of the medium in a well while the lid is in place.

According to a further embodiment, the invention provides a system for culturing and transporting embryos comprising the device of the invention and an appliance or transport module which operates in conjunction with the device, the appliance or module comprising: one or more fluidic reservoirs for supplying fluid to the device, fluidic connection means to effect fluidic communication between the appliance and the device, flow generation or control means to effect or regulate flow on the device, a power supply to allow operation of the device and the appliance independently of an external power supply, a control means to control operation of the device and the appliance, optionally using the output from sensors associated with the device.

The surface is preferably flat or planar. The wells preferably form a two dimensional array for ease of automatic insertion of cellular entities or microscopic examination.

Preferably the apparatus is arranged to give visibility of the embryo in a well for observation through the base, using an inverted microscope, from above, using a standard microscope, or both.

Preferably means to control the temperature of the medium in the well are provided.

Preferably one or more temperature sensors to measure the temperature of the apparatus itself or the medium in the well is provided.

Preferably one or more heat transfer means to heat or cool the apparatus itself or the medium in the well is provided.

In one embodiment all or part of the device is made from a gas-permeable but liquid-impermeable material such as PDMS. PDMS has a high solubility for gas and a low solubility for aqueous liquids and so can sustain sufficient transport of oxygen and CO₂ across a suitable thickness of the material for metabolism of cellular contents of the wells. The components are sized to allow sufficient transport rate through the bulk material that respiration of the cellular contents of the wells is sustained.

In an alternative preferred embodiment, the lid or base is made from a porous hydrophobic material that supports gas transport but does not allow access of aqueous liquid into the pores. Such materials exist in several forms, but one found particularly suitable is porous sintered polypropylene, trademarked as ‘VYON’ and supplied by Porvair Ltd., Wrexham, UK. This material is structurally robust and has high gas transport coefficients.

In preferred embodiments the base is thin, to allow good optical properties when placed on an inverted microscope, and also to give good thermal contact between the contents of the wells and the lower surface of the base, so allowing close temperature control of the contents when the device is placed on a heating or cooling surface.

Preferably at least part of the surface of the lid and/or the base is hydrophilic.

Preferably at least part of the surface of the lid and/or the base is hydrophobic.

Preferably a controlled release device which acts to release substances into the well is provided. The controlled release device may be autonomous, for example time-release, or controllable, for example using an external control signal or stimulus.

Preferably one or more fluidic channels in fluid communication with the well, through which medium or gas may flow are provided.

Preferably a supply of material to be added to the medium in the well, so as to change the chemical composition of medium in the well is provided.

Preferably means to allow gaseous communication between the wells and a supply of gas, either in the environment immediately surrounding the apparatus or supplied via a further fluidic channel is provided.

Preferably a gas reservoir in fluidic communication with a permeation means located on the device or as part of the fluidic flow system of the appliance, which permeation means allows transport of gas molecules from the gas reservoir to the medium in the device is provided.

Preferably thermal insulation is provided between the device and the environment.

Further temperature sensors preferably are provided that measure the temperature of the appliance or its external environment, the output of which is logged or utilised by the control means, for example to control heat transfer means or flow within the appliance or device.

Preferably at least one further sensor, for example dissolved oxygen and/or pH sensors, which monitors conditions either in medium in the well or in medium in fluid communication with it is provided.

Preferably one or more of the following is provided: —data logging means that records data from the sensors of the system, such as the temperature, pH, dissolved oxygen or other sensors as described above associated with conditions in the medium to which the cellular entities are exposed; sensors elsewhere in the system, such as internal and external temperature sensors which measure the correct functioning of the system and the environmental conditions in which it is located; accelerometers and attitude sensors which might be provided to detect motion or untoward events; communication means that allows communication between the appliance and a remote system, such as a mobile telephony interface or a wireless data interface; GPS position monitoring means; which together can act to monitor or control the operation of the appliance and the device, log its position and report status and positional information to a remote station.

The aforementioned preferred features may be provided as part of the device or as part of the apparatus or appliance.

In a further embodiment, the transport system of the invention further comprises means for stabilization of the temperature of the inside of the apparatus and or the device, comprising:

a thermally insulating outer housing comprising a receiving region for a heat sink such as a cold body
a heat sink maintained at a temperature below that at which the temperature is to be stabilized
a thermally insulating region between the heat sink and the device
means to sense the temperature of the device or its surrounding region and supply heat to the device
control means to control the temperature of the device in response to sensor inputs.

In a preferred embodiment the heat sink comprises a cold body, comprising a material or assembly which may be cooled before introduction into the apparatus.

In another embodiment the heat sink comprises a heat exchanger which acts to dissipate heat to the outside of the outer insulating housing.

In a preferred embodiment the device and heat supply means are located within a closed thermally inner insulating region outside which the cold body is located.

In a preferred embodiment the cold body is distributed substantially around the inner insulating region.

In a preferred embodiment the cold body comprises a phase change or eutectic material, for example a gel, which is adapted to absorb or release latent heat at a temperature below that at which the device is desired to be held.

The device heater and control means regulate the amount of heat needed to keep the device at a set temperature above the temperature of the cold body. The power input to the heater is controlled by the control means in relation to the rate of heat loss through the insulation to the cold body.

In an alternative embodiment, the cold body may be any other material which is suitable to be pre-cooled in a freezer or refrigerator, and which can be mounted into the apparatus before shipping. Such a material may be liquid or solid, preferably contained within a subcomponent designed for ready handling and ease of mounting in the apparatus.

In a preferred embodiment the system additionally comprises means to monitor and the temperature of the region in which the device is to be placed, before and after the cold body has been mounted in the device, to ensure that the device experiences a controlled temperature profile.

In a preferred embodiment the apparatus comprises one or more temperature sensors which sense the temperature of the cold body and which are read by the control means. The output from this sensor may then be used to monitor the status of the transport module and to control the heating supply means.

In a preferred embodiment the control means comprises a program which acts to:

sense the temperature of one or more of: the transport module, the environment outside the outer insulating housing, the region inside the outer insulating housing, the region inside the inner insulating housing, the device, the medium within the device, any reservoirs for medium that are provided within the apparatus, and the temperature of medium within the apparatus.
control heating and/or cooling means in response to the sensory input so as to control a temperature or temperature profile according to pre-programmed or subsequently communicated instructions
log and optionally communicate the status of the transport module during transit.

The wells for the cellular entity can be of any form provided that they form a designated area for retaining the cellular entity.

The invention will now be described, by way of example only, with reference to the accompanying schematic figures, in which:—

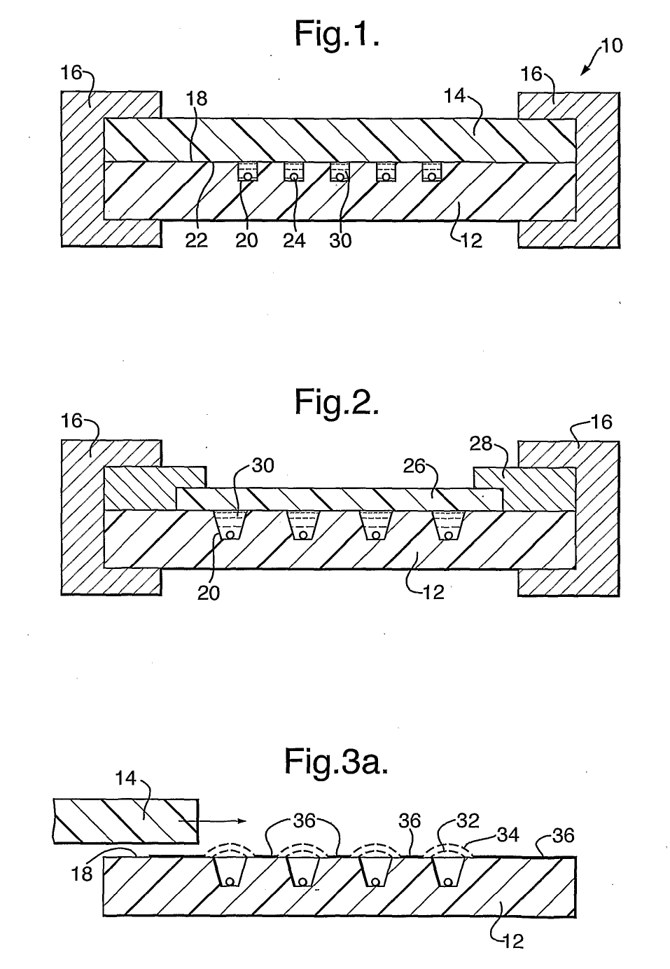

FIG. 1 shows a vertical cross-section of a device according to a first embodiment of the invention;

FIG. 2 shows a vertical cross-section of a device according to a second embodiment the invention;

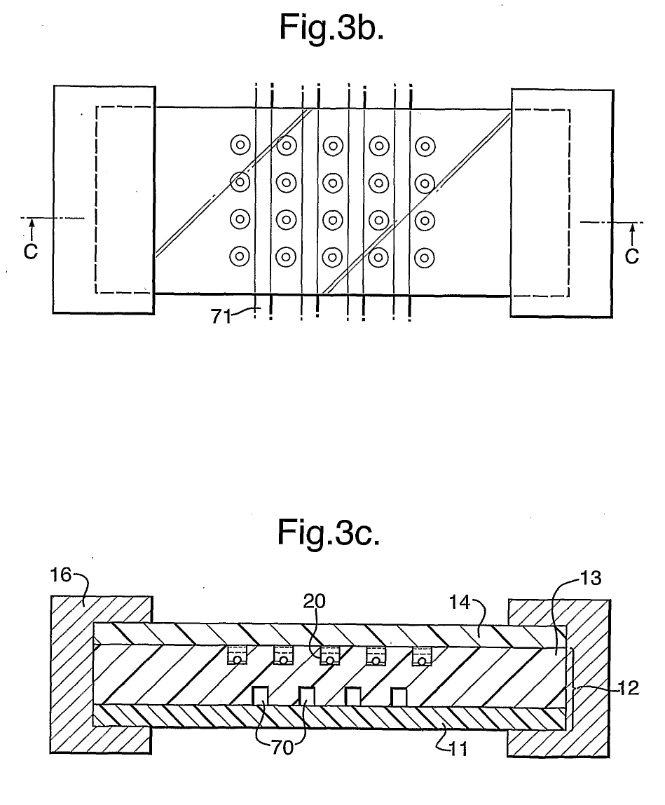

FIG. 3a shows a vertical cross-section of the device of the second embodiment, showing a method of applying the lid to the device;

FIG. 3b shows a plan view of the device of a further embodiment;

FIG. 3c shows a vertical cross-section along line C-C in FIG. 3b;

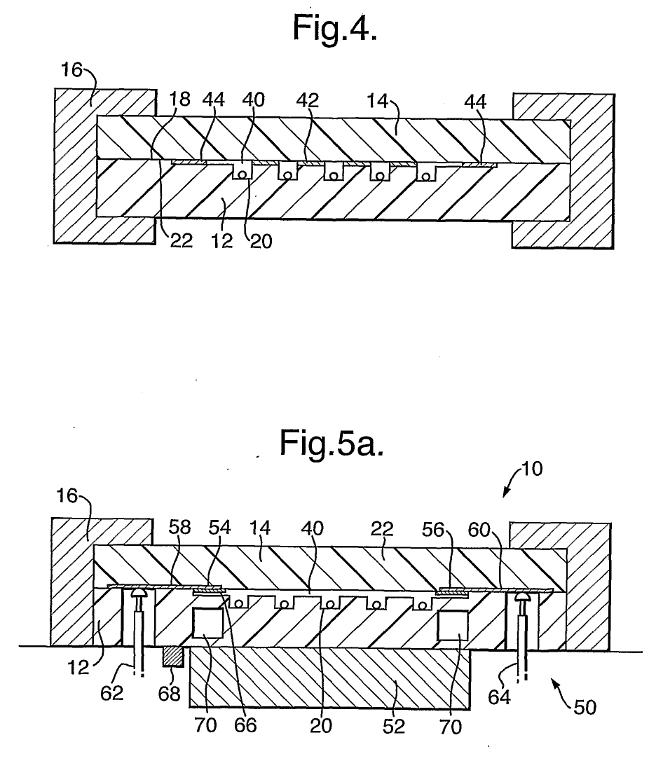

FIG. 4 shows a vertical cross-section of a device according to a third embodiment of the invention;

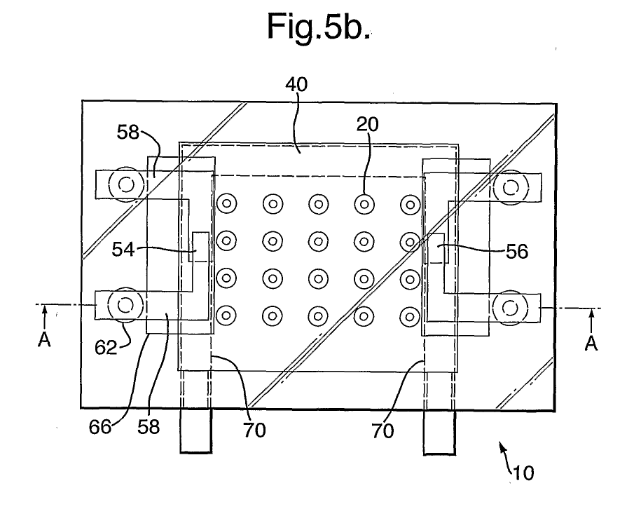

FIG. 5a shows a vertical cross-section of a device according to a fourth embodiment of the invention;

FIG. 5b shows a plan view of the embodiment shown in FIG. 5a;

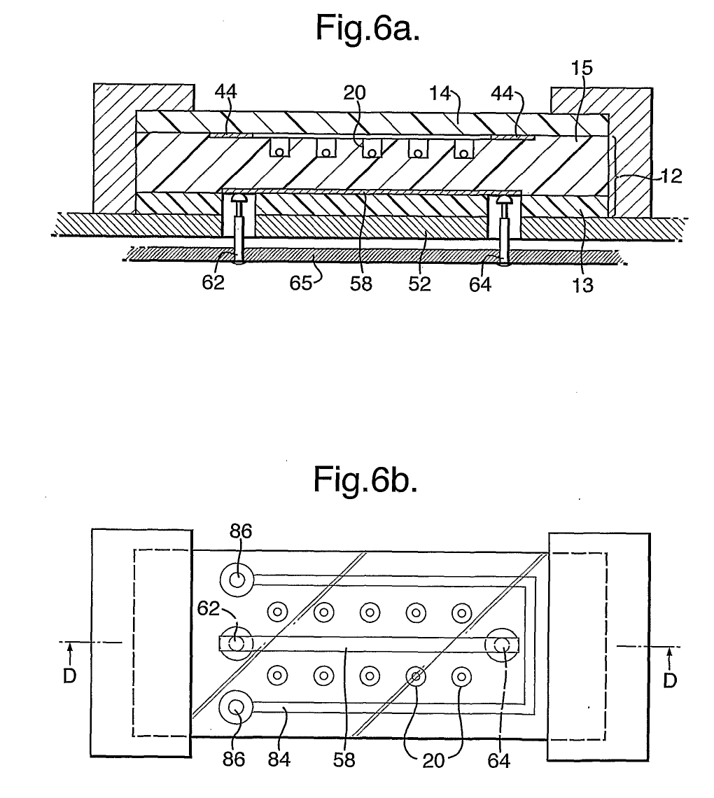

FIG. 6a shows a vertical cross-section of a device according to a fifth embodiment of the invention;

FIG. 6b shows a plan view of the embodiment shown in FIG. 6a;

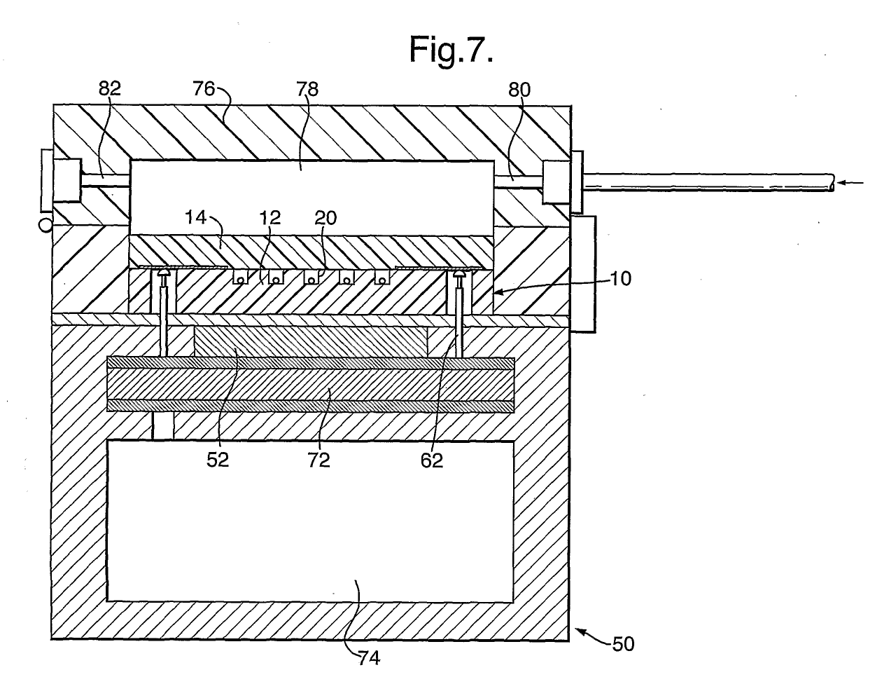

FIG. 7 shows a schematic vertical cross-section of a system of the invention, comprising a device and an appliance;

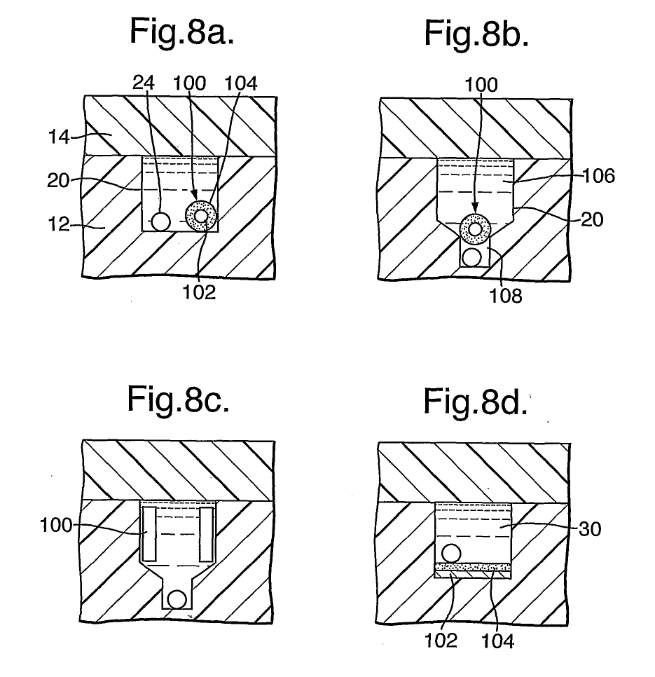

FIG. 8a shows a vertical cross-section of a well of a sixth embodiment of a device of the invention;

FIG. 8b shows a vertical cross-section of a well of a device according to a seventh embodiment of the invention;

FIG. 8c shows a vertical cross-section of a well of a device according to an eighth embodiment of the invention;

FIG. 8d shows a vertical cross-section of a well of a device according to a ninth embodiment of the invention;

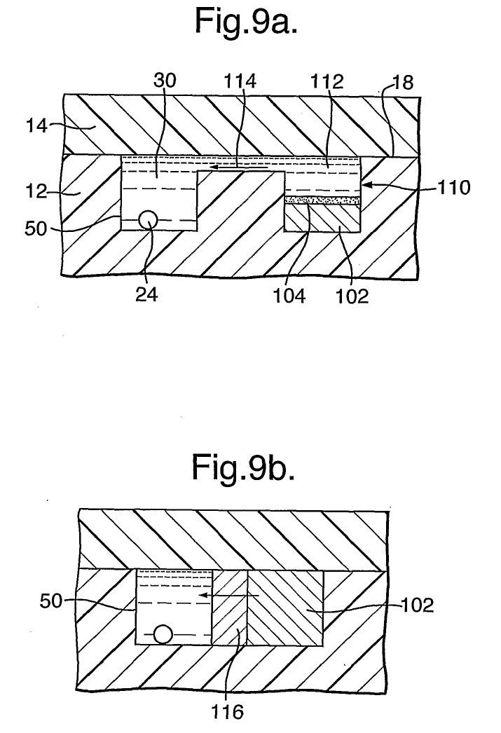

FIG. 9a shows a vertical partial cross-section of a device according to a tenth embodiment of the invention;

FIG. 9b shows a vertical partial cross-section of a device according to an eleventh embodiment of the invention;

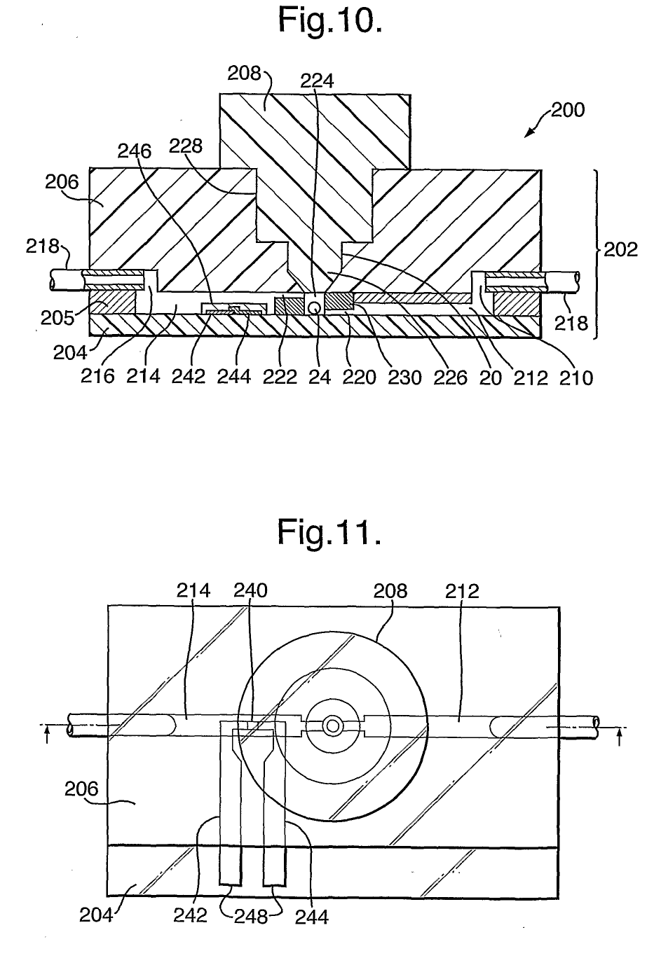

FIG. 10 shows a vertical cross-section of a device according to a twelfth embodiment of the invention;

FIG. 11 shows a plan view of the embodiment shown in FIG. 10;

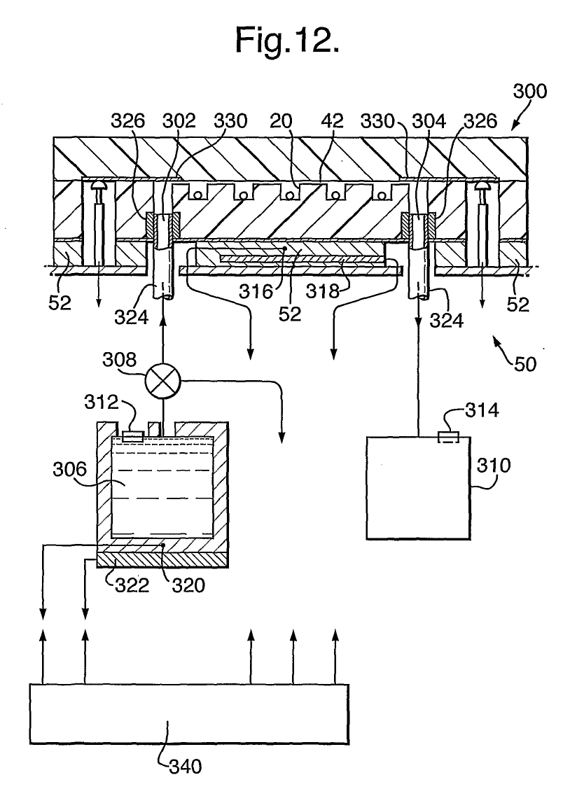

FIG. 12 shows a vertical cross-section of a device according to a thirteenth embodiment of the invention, together with a schematic diagram of a fluid flow system of the appliance of the invention;

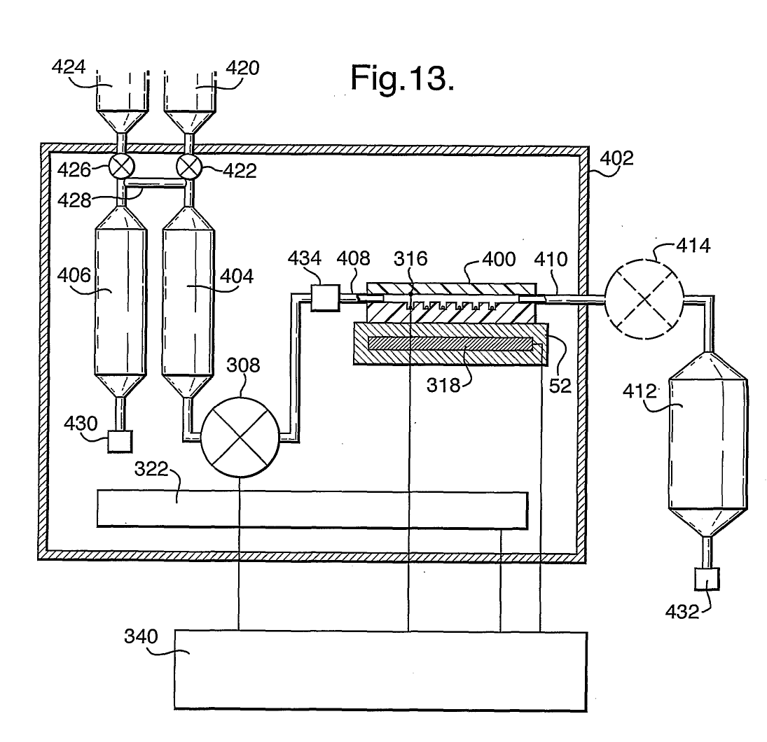

FIG. 13 shows a schematic diagram of a system of the invention, comprising a device and an appliance, with elements of a fluid flow system for use in the appliance; and

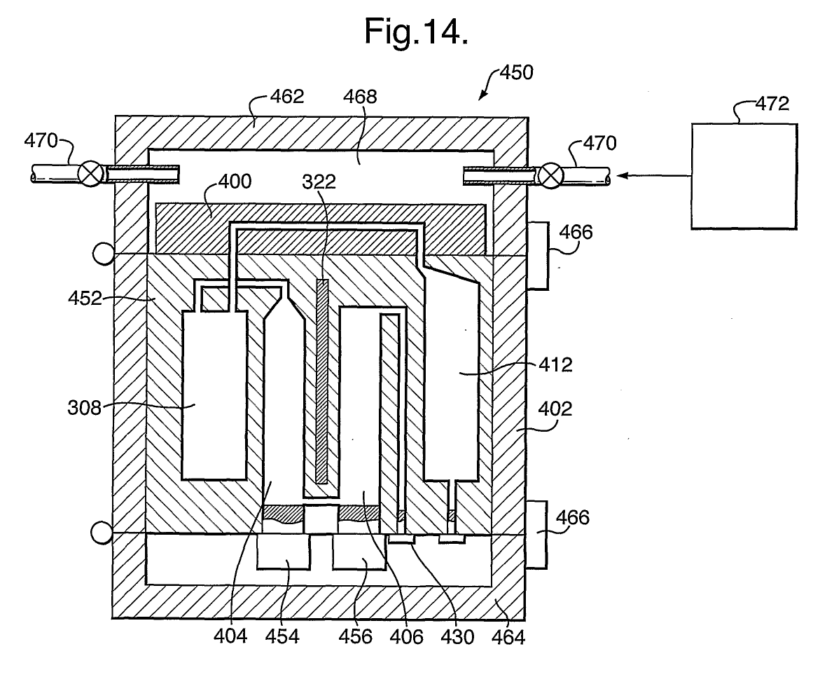

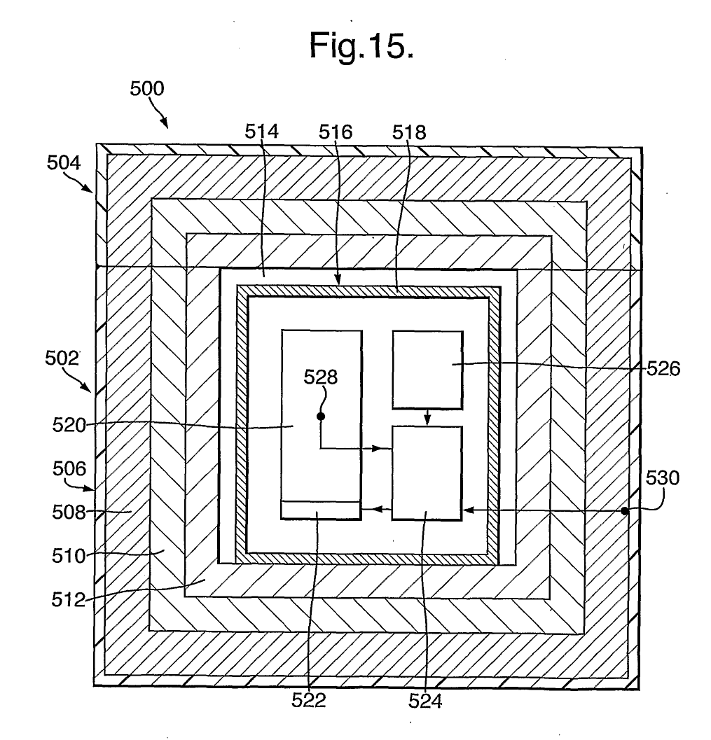

FIG. 14 shows a schematic diagram of the system of FIG. 13, comprising a device and an appliance, with elements of a fluid flow system for use in the appliance, whilst FIGS. 15 to 22 show further vertical cross-sections of embodiments of the present invention.

FIG. 1 shows a device 10 comprising a base 12 and a lid 14, held together by one or more clips, clamping means or other retaining devices 16. The base 12 has a surface 18 in which are formed one or more wells 20 sized to accommodate the objects of interest, shown as 24 in FIG. 1, bathed in medium 30. The lid 14 has a surface 22 that seals against the surface 18 when the lid is assembled onto the base so retaining the contents of the wells 20. The wells 20 may be of any suitable form to accommodate the objects—they are shown in FIG. 1 as having straight sides and flat bases but equally they may be tapered or stepped and have rounded bases, and may be of any cross-sectional shape. The device 10 is adapted to allow exchange of gas with the external environment. In one embodiment all or part of the device is made from a gas-permeable but liquid-impermeable material such as PDMS. PDMS has a high solubility for gas and a low solubility for aqueous liquids and so can sustain sufficient transport of oxygen and CO₂ across a suitable thickness of the material for metabolism of cellular contents of the wells. In an alternative preferred embodiment, the lid or base is made from a porous hydrophobic material that supports gas transport but does not allow access of aqueous liquid into the pores. Such materials exist in several forms, but one found particularly suitable is porous sintered polyethylene or polypropylene, for example the material trademarked as ‘VYON’ and supplied by Porvair Ltd., Wrexham, UK. This material is structurally robust and has high gas transport coefficients. In one embodiment therefore the lid 14 is formed entirely from the porous hydrophobic material, this having sufficient strength to allow it to be held sealed against the base 12, so as to seal the wells 20 against loss of liquid contents while allowing gas diffusion through the lid to the interior of the wells. In alternative embodiments, for example as shown in FIG. 2, the lid may comprise a subcomponent 26 formed from the gas-permeable but liquid impermeable or porous hydrophobic material held in a rigid non-porous main component 28.

Optionally the base and lid are held together without retaining devices, for example by means of tight interfitting or adhesion between regions of the lid and the base.

The device 10 itself may be of any size, suitable to accommodate any number of wells 20. The device is advantageously formed to a standard size to interact with standard biotechnological equipment, such as microplate handlers or microscope slide holders.

The wells 20 may be sized to contain a large volume of medium per object as in FIG. 2 or a smaller volume as in FIG. 1. One or more objects may be housed in a well. A tapering profile in the well 20 as in FIG. 2 is advantageous in some embodiments to locate an object sedimenting into the well, following pipetting, to a central location in the base of the well for easier visualisation, especially if the well is large and visualisation is to be done automatically or semi-automatically. Preferably the base 12 is formed from a transparent material to allow visualisation through the base of the wells with an inverted microscope. The lid material may be transparent or translucent to allow illumination from above. In a preferred embodiment the lid is formed from porous sintered polymer, which is translucent, and the base is formed from polystyrene, acrylic or another polymer that is transparent. The thickness of the base 12 below the base of the well can be chosen to suit the optics used for observation.

In preferred embodiments the wells 20 are sized to accommodate the objects of interest, while containing an amount of medium that is small compared with similar apparatus of the prior art. Typically the wells will have a volume between 1E-6 μl and 100 μl, and a typical minimum dimension in the range 10 μm to 5 mm. More preferably, for objects such as embryos, oocytes and cumulus-oocyte complexes, with typical dimensions in the range 50 to 500 μm, the wells will have a volume in the range 1E-3 μl and 100 μl and a minimum dimension in the range 100 μm to 5 mm. For culture of large numbers of other cells in common medium space these dimensions will also be suitable, but for culture of smaller numbers of cells the preferred dimensions are smaller, with well volume in the range 1E-6 μl and 1 μl, with minimum dimension in the range 10 μm to 1 mm.

In preferred embodiments the base 12 is thin, to allow good optical properties when placed on an inverted microscope, and also to give good thermal contact between the contents of the wells and the lower surface of the base, so allowing close temperature control of the contents when the device is placed on a heating or cooling surface.

In use, the well 20 is filled with medium 30 and objects 24 deposited into it, either manually or using a robotic pipettor. Multiple wells 20 may be formed to a standard format and arranged on a standard grid, such as the SBS microwell plate standard, to allow easy interface to robotic pipetting equipment. The base and lid are adapted to allow easy application of the lid to the base without trapping an air bubble in the well. This can be done as in standard practice using microscope slides and cover slips by arranging for at least part of the surface of the lid and base to be hydrophilic, so allowing the medium to wet the surface and a sliding motion to displace excess medium over the surface of the lid and base before they seal. In a preferred embodiment shown in FIG. 3a the surface 18 of the base 12 is made hydrophobic in at least part of its area, so as to allow menisci of medium 34 in the well to stand proud of the surface when the wells are filled to that level, such as in position 32 in FIG. 3a. The lid 14 may then be applied so as to intersect the menisci and break the surface tension in such a way that bubbles are not trapped in the wells once the lid is in place. The surface 18 may be wholly hydrophobic, or the hydrophobicity may be partial or patterned, as indicated at 36 in FIG. 3a. Menisci may then intersect the surface 18 at the junction between the hydrophobic and hydrophilic regions as shown.

The clip means 16 is shown as a simple spring clip in FIGS. 1, 2, 3b and 3c and indeed such a simple clip may be used with the device, the clip being applied by hand after the lid has been put in place. Other forms of clip or clamp, such as press clamps or electromagnetic clamps as known in the art may be used, in particular if the device is to be used with automatic liquid and/or microplate-handling equipment.

FIG. 3b shows a plan view and FIG. 3c a cross-section at C-C on the plan of a further embodiment similar to those in FIGS. 1-3a, in which exchange between a gas environment and the medium in the wells is facilitated by one or more gas supply channels 70 formed in the base, close to the wells so as to allow ready diffusion of gas molecules through the material of the base. This embodiment is particularly advantageous when the wells and the gas supply channels are formed in PDMS. In a preferred embodiment, the base 12 comprises a substrate 11 and a body component 13 preferably formed from PDMS or another polymer of high gas permeability. The substrate may extend over the whole or part of the body component, and may be a subcomponent of the base rather than forming a structural component. For example, the substrate is glass or polycarbonate and the PDMS layer is plasma-bonded to it as known in the art. The gas exchange channels may simply be open to a surrounding gaseous atmosphere, or may be joined by one or more fluidic connectors 71 to a supply of gas. A number of discrete channels may be used as in FIGS. 3b and 3c, or a smaller number of channels may be used which lead past a greater number of wells, in some embodiments with a serpentine pattern. The channels may be formed through the thickness of the substrate 11 opening at its major surface, or may extend through the body component 13 opening at its major surface.

FIG. 4 shows a further preferred embodiment of the invention, in which the wells 20 are in fluid communication with a common fluid space 40, defined between the surface 18 of the base and the surface 22 of the lid. The lid may seal to the base using an optional seal means 44 surrounding the space 40. The regions 42 between the surface 18 and 22 that lie between neighbouring wells are diffusion paths between the wells, and are adapted so that objects in neighbouring wells cannot leave the wells and come into contact, but are in chemical communication via the fluid in the space 40. The regions 42 might simply be parts of the space 40 narrow enough to confine the objects. In another preferred embodiment, the regions 42 are occupied wholly or partially by permeable material that allows diffusive transport, for example a hydrophilic porous material which is wetted by the medium but has pores too small for the objects to pass through. Such a material also advantageously acts to restrict physical flow of the medium in the case that the device is moved or shocked or experiences a temperature gradient, and so is advantageous in a device intended for transportation of the objects. A suitable material has been found to be porous sintered polypropylene treated to render it hydrophilic. An example is VYON™ by Porvair Ltd., as cited above. Other materials, which are permeable rather than porous, are also applicable, for example hydrogel polymers which can be formed on the surface of the lid to the intended pattern, or on the surface 18 of the base between the wells, by means known in the art. In an alternative embodiment, the wells themselves are formed in the permeable polymer, such as a hydrogel.

The device of FIG. 4 can be sized to suit the objects in the wells and the intended degree of diffusional connectedness between the wells. The spacing between the wells, the depth of the space 40 and the regions 42, and the diffusional properties of material present in the regions 42 (if any) will all control the diffusional intercommunication between the wells and so can be chosen to suit the intended purpose. In the case that the device is used for cells rather than embryos, use of a diffusion-limiting material in the regions 42 is particularly advantageous as it relaxes the constraint on the height of the space 42 being less than the minimum dimensions than the cell.

In a further embodiment, the material in regions 42 is chosen to be active, i.e. to change over time and/or in response to its environment. For example, the material is chosen from the group of slowly-hydrating hyrodgel polymers, whose diffusional properties change with hydration, the diffusion coefficient increasing with increasing degrees of hydration. In this embodiment the wells are initially isolated one from another, and are increasingly diffusionally connected as time goes on. This is potentially advantageous in circumstances where the conditions are intended to change during transportation, from culture of isolated objects to joint culture, and in particular when the composition of the medium is being changed to progress culture while in transit. Similarly, a slowly-dissolving material in the regions, such as a less cross-linked gel composition, would open the diffusional pathway over time. Provision of a hydrogel layer that at first only partially fills the region 42, but which swells gradually with time, would steadily restrict diffusional interconnection should that be desired.

FIG. 5a shows a further preferred embodiment, in which means are provided to monitor and/or control the temperature of the device and contents of the wells. The device is substantially as in the embodiment shown in FIG. 4 but with the following additional features, which may also be included in devices of the present invention, for example as shown in the other figures. The device is shown located at a location site on an appliance 50, in contact with a heat exchange means 52 that acts to heat and/or cool the device. The retaining devices 16 are shown diagrammatically and can be of any form appropriate to maintain good thermal contact between the device and the heat exchange means. The device is provided with one or more sensors in contact with the medium in the wells 20 or the common space 40, or in such proximity to it that they can sense the conditions in the medium. In FIG. 5a two temperature sensors 54, 56 in the form of thin-film thermocouples or resistive thermometers are shown formed on the surface 22 of the lid 14. These are connected by two or more contact tracks 58, 60 to external contact means shown in the form of spring pins 62, 64 which make electrical connection between the device and the appliance 50. In the case of thin-film metal temperature sensors, it is important to isolate the conducting elements from the medium, so a thin insulating coating 66 is provided over at least the conductive regions exposed to the medium. The one or more temperature sensors on the device allow close feedback control of the temperature of the medium using a control means (not shown) in conjunction with the heat exchange means.

In an alternative embodiment the temperature sensors are provided mounted on or associated with the base 12. The sensor might be located on the surface 18 of the base, or within the material of the base at a short distance from the bottom of the wells or the space 40.

Further, one or more temperature sensors 68 could be mounted on the base or lid of the device, so monitoring its outside temperature. If the device is located in use in a closed, insulated environment then this can be designed to be effectively isothermal, and the external device temperature will be a good approximation to the temperature of the medium.

In FIG. 5a the heat exchange means 52 might be an electric heater, a peltier device, a metal block heated or cooled by fluid flowing through it. It might be a passive means used to maintain an even temperature over the base 12 of the device, both the device and the means 52 being heated by a heat source such as flowing air. In the case that a peltier device is used, the means 52 might comprise additionally heat transfer or heat sink means which conveys heat to or from the external environment, such as a heat pipe or external radiator as known in the art.

Further, or in the alternative, one or more fluid flow passages are provided either wholly or partially defined by the material of the base and/or lid, through which fluid may flow to maintain the temperature of the device. For example, fluid flow passages may be defined within the base material as indicated in cross-section at 70 in FIG. 5a. Such flow passages might have a serpentine form through the base of the device so as to bring them into close proximity with the wells, or might flow around the perimeter of a group of wells. The fluid is preferably maintained at a constant temperature by a heater remote from the device, controlled by the appliance 50.

FIG. 5b shows a plan view of the embodiment shown in FIG. 5a, with the assumption that the lid is of transparent material so that the interior of the device can be seen when the lid is in place. For clarity the clamps 16 are not shown. FIG. 5a is a cross-section corresponding to A-A in FIG. 5b. A 5×4 array of 20 wells is shown. It will be understood that any number or configuration of wells is within scope of the invention. The temperature sensor 56 is shown, visible through the lid material, along with contact tracks 58 making contact with the contact means 62, seen from above. A further feature in certain embodiments is a fluid flow channel 70 through which heating or cooling fluid may be passed, shown dotted in FIG. 5b. Preferably such channels 70 will not pass directly under the base of the wells, to retain good visibility from below. The pattern that such channels may have will be determined by the need for even heat flow to give uniform temperature distribution. Therefore a preferred arrangement will be serpentine, leading close to or through the well area, preferentially between the well axes rather than crossing them.

FIGS. 6a and 6b show a further embodiment of a device in which electrical connection is made to the device from an appliance. FIG. 6b is a plan view of the embodiment and FIG. 6a is a cross-section at D-D in FIG. 6b. Contacts are made using spring contacts 62, 64 as before, to a temperature sensor in the form of a resistance thermometer 58 formed or mounted on the base of the device and located adjacent the wells. The sensor 58 is preferably located to one side of the wells as shown in plan view in FIG. 6b—the representation in FIG. 6a is to show a typical vertical position of the sensor relative to the wells and the base component, and to illustrate a practically useful structure for the base 12, that is formed from two or more subcomponent layers 13 and 15, on one of which metal tracks for contacts, sensors or other components can be formed or mounted. In FIG. 6b a heater track 84 is shown, running close to the wells and preferably arranged so as to give an approximately even energy density over the area of the device. Contact can be made to the heater track using further standard contact means 86. It will be appreciated that the arrangement of the heater track and one or more sensors can be varied to suit the layout of wells on the device.

FIG. 7 shows an embodiment of a system of the invention, comprising a device 10 formed from a base 12 and a lid 14, and an appliance 50 adapted to allow transport of the device under controlled conditions, on or in which the device is located. The appliance comprises a heat exchange means 52, one or more electrical contact means 62, a control means 72 which receives signals from sensors associated with the device and/or the appliance and acts to control the operation of the device and/or the appliance and a power source 74. In preferred embodiments the appliance comprises a gas reservoir which is, or can be brought to be, in fluid communication with the device and which can act as a reservoir of gas which can be exchanged with dissolved gas in the medium while the appliance and device are remote from external gas sources. The gas reservoir might operate at atmospheric pressure or might be at over-pressure. In FIG. 7 the appliance has a lid 76 comprising a gas reservoir in the form of a gas space 78. The lid can form a gas-tight seal around the device, the lid having one or more ports 80, 82 which allow flushing of the space with gas (typically 5% CO₂/air) and isolation of the gas within the space when the lid is closed.

The above embodiments serve to retain single or groups of objects in fixed locations in controlled volumes of medium with optional diffusion between the objects. In further particularly preferred embodiments the device is adapted to change the composition of the medium bathing the objects as a function of time or in response to an external stimulus.

FIGS. 8a to 8d show embodiments of the invention in which the composition of the medium in a well is changed by operation of a separate timed-release structure within, or in fluid communication with, the device, for example within one or more of the wells on the device. In all the embodiments in FIGS. 8a to 8d the remainder of the device (not shown) and appliance that can be used in a system with the device is according to any of the embodiments described herein.

FIG. 8a shows a first embodiment comprising a controlled-release structure 100 in a well with an object 24. The structure 100 here comprises an inner core of material 102 which is to be added to the medium surrounded by a slowly-dissolving coat material 104 (such as, for example, a sugar). Such multilayer compositions are well known in the art of drug-delivery and a number of suitable materials and vehicles are available. The material 102 may itself be active in the culturing process, may act to bind and remove from availability a substance in the medium, or both. Of course, if release is to be started immediately on adding medium the coating material 104 can be omitted. FIG. 8b shows a well with a tapered or stepped cross-section that acts to locate the object in a first part of the well and the structure 100 in a second part of the well. In FIG. 8b the object is located in a narrow lower part of the well 108 while the structure 100 is retained in the wider upper part 106. FIG. 8c shows a well of a further embodiment in which the structure 100 is formed instead into a shape that is designed to locate in a certain part of the well so giving a defined geometry of the release process relative to the well and the object. In FIG. 8c the structure is disc-shaped and is retained in an upper part of the well while the object sediments to the bottom. Such a structure might be formed from an insoluble body part with a soluble layer or closure, which is breached with time so releasing the contents. FIG. 8d shows a further embodiment in which the material 102 is deposited in the base of the well, covered by the release controlling material 104 in an upper layer. In this embodiment the wells 30 are prepared before filling with medium to programme the material 102 and the timing of release by the composition and thickness of the coat material 104.

Other designs of release structure will be apparent to those skilled in the art and may be used in the device and method of the inventions. In particular, the substance to be released can be covered wholly or partly by a barrier material, such as for example a hydrogel, which slowly expands on contact with a liquid to become permeable.

FIGS. 9a and 9b show two further embodiments in which controlled release is achieved by pre-prepared structures that are formed as part of the device 10. In FIG. 9a the device comprises a reservoir 110 itself comprising material 102 to be added to the medium in the well 50. The reservoir is in fluid communication with the well through a flow path 114 which may be defined to pass through the interface between the base 12 and the lid 14, or may be formed through the body of the base itself. The reservoir is advantageously in the form of a well open to the surface 18 of the base, into which medium may be pipetted before the lid is fitted. The material 102 might be alone in the reservoir or might be covered or mixed with release controlling material 104 that acts to delay the dissolution of the material 102 into the medium 112 in the reservoir. Once material 102 has dissolved it is free to diffuse through the fluid pathway 114 and into the well 30. The process of addition of material 102 to the medium in the well 30 will be timed by the material 104, the dimensions of the reservoir and the fluidic pathway. In general diffusion is a slow process, but that is in general what is required in changing a culturing medium and is in fact an advantageous feature of this embodiment of the invention. The fluidic pathway 114 might be a passage linking the reservoir and the well, or might by wholly or partly filled with a material which controls diffusion and/or convection, such as a hydrophilic porous material, hydrogel or similar as described for the embodiment in FIG. 4 above, and might also be active in that its properties change with time to increase or to decrease diffusion. For example, control material 104 might be present in the space 114. FIG. 9b shows a further embodiment in which the reservoir is adjacent the well and linked to it by a porous or permeable element 116 through which the material 102 gradually diffuses. The timescale of addition is now controlled by the material 116, and the geometry of the arrangement allows a more uniform introduction of the material into the well 50.

FIG. 10 shows a device according to a further embodiment of the invention, adapted for culture and transport of objects in which the medium in the well can be changed while the object is retained in the well against flow, physical movement and shock. In FIG. 10 a single well and associated flow channels are shown but it will be appreciated that in other preferred embodiments multiple wells, each part of a fluidic pathway formed from channels and other features as in FIG. 10, are included in the same device and optionally are supplied with fluid from one or more common fluidic channels.

The device 200 comprises a base 202 and a lid 208, the base optionally being formed from a substrate 204, a first body part 205 and a second body part 206 permanently bonded together. The base comprises a well 20 as before adapted to contain an object 24. The lid 208 is removable to give access to the well and when in place seals a fluidic path through the device, comprising an inlet port 210, an inlet channel 212, the well 20, an outlet channel 214 and an outlet port 216. The inlet and outlet port are shown in FIG. 10 as leading to the exterior of the device via connection means 218. Alternatively they may be in fluid communication with one or more further fluid channels formed as part of the device, which in a preferred embodiment lead to other flow systems similar to that shown in FIG. 10. The device might also comprise one or more fluid reservoirs for supplying fluid to, or receiving fluid from, the inlet and outlet ports of each flow system as shown in FIG. 10. The fluid flow pathway is reversible—the inlets referred to here may be used as outlets and vice versa. The object 24 is retained in the well by a first constriction region 220 formed in the inlet channel near the base of the well that acts to prevent the object from leaving the well, and a second constriction region formed in the pathway at the exit from the well, shown in FIG. 10 as being defined by the first and second body parts 205, 206, but which might in other embodiments be formed between a surface of the base and a surface of the lid. In a preferred embodiment, as shown in FIG. 10, the well 20 has a tapered or stepped profile with an inner region 224 of smaller cross-sectional dimension and an outer region 226 of larger cross-sectional dimension. One of the lid and the base body component 206 are made of a compliant material, allowing a tight fit between the lid and a larger recess 228 provided in the base, so retaining the lid in place without the need for an external fixture. The portion of the lid that fits into the well then acts to close the well.

The device 200 of FIG. 10 may be formed by bonding the substrate 204 onto body parts 205, 206 made by moulding or machining, with the features of the well and flow path defined by the mould. For example, the body parts may be moulded from PDMS and the substrate be glass or a polymer, the body parts being bonded to the substrate by plasma activated bonding as known in the art. The body parts may be made for example from PDMS and bonded together. In a preferred embodiment one or both of the first and the second constriction is defined wholly or partially by a separate moulded component that is inserted into the body part 206. One or both of the constrictions may be defined within the insert 230 and one or both may be defined by a space between the insert and the substrate 204, the first body part 205 or the second body part 206. In this embodiment, the first body part indicated as 205 is reduced to an insert 230 shown as cross-hatched in FIG. 10—the remaining parts of the first body part 204 being incorporated into the second body part 206. One or both of the constrictions may be defined within the insert 230 and one or both may be defined by a space between the insert and the substrate 204, the first body part 205 or the second body part 206. In an alternative embodiment, the substrate, first and second body parts are moulded from rigid material, such as acrylic or polycarbonate, and laminated, pressure bonded or adhesive bonded together. In embodiments where the second body part 206 is of compliant material, the lid may be moulded from any rigid polymer, such as acrylic. In embodiments where the second body part is rigid, the lid may be moulded from a compliant polymer, e.g. PDMS.

This form of construction has the advantage that the resulting device is optically transparent and provides observation through a good quality planar substrate using an inverted microscope.

FIG. 11 shows a plan view of an embodiment according to FIG. 10, showing the plan at the level of the substrate 204. An optional sensor 240 is shown, formed or mounted on the substrate 204, and connected to contact terminals 248 by tracks 242, 244. The sensor is optionally a temperature sensor, and may be formed as a thermocouple from two contacting metals, or as a resistance thermometer with a single metal, in both cases isolated electrically from medium in the flow channel by a thin overlayer 246 (see FIG. 10). The sensor is shown in the outlet channel 214 but may equally be located elsewhere in the device, either in proximity to a flow channel, the well, or away from these. More than one sensor may be provided. The sensor might also be other than a temperature sensor—for example a sensor for dissolved O₂, or for pH, in which case the overlayer 246 may be active to control access of species to be sensed to the metal electrodes 242, 244, or to act as an electroactive membrane to sense the property desired. Overlayer 246 might be a polymer whose resistance changes in response to pH, or might be an electroactive membrane, the potential across which will change in response to pH or other ion concentration. In this case a multilayer structure may be formed in the sensor region over the electrodes as is known in the art.

In preferred embodiments there are multiple wells and associated flow systems as part of the device, in which case FIG. 11 represents a partial plan view of the device. The tracks 242, 244 and the contacts 248 can be formed at any location on the device.

FIG. 12 shows a device according to a further embodiment of the invention, and a schematic diagram of elements of an appliance of the invention. In this embodiment the device 300 comprises one or more wells 20 in fluid communication with a fluid space 42, this space forming part of a flow path through the device from an inlet port 302, through the space 42, to an outlet port 304. The flow path brings fluid flowing along it into contact with medium in the wells 20, allowing substances in the wells to exchange with substances in the flowing fluid in the space 42. This allows renewal of the medium in the wells or change in its composition, according to which fluid is flowed into the inlet port. The device is preferably provided with one or more temperature sensors 330, located so as to be in thermal contact with the medium in the space 42 and wells 20, connected via tracks and contact means as previously described. The device is located at a location site on or in the appliance 50, in contact with heat exchange means 52, here shown as a heater block comprising a temperature sensor 316 and a heater 318. Fluidic connection is made to the device by two or more connectors 324, here shown as being a push-fit into connector means 326 on the device, but which may take any appropriate form, including conventional Luer or screw-fit HPLC connectors and ‘flying-lead’ tubing.

The appliance 50 comprises fluid supply and flow means for operation in conjunction with the device, comprising one or more fluid reservoirs 306, pump means 308, waste reservoir 310, the reservoirs being equipped with breathers 312, 314 to equalise pressure. More than one reservoir 306 may be provided, each with a different medium, either connected in series in the flow path so that the contents of one flows substantially completely through the flow path before the contents of the other starts to flow through the path, or with valve means to select which reservoir is connected to the flow path. The pump then flows medium through the flow path and exchanges medium with wells 20 according to a pre-set programme or to conditions detected in the device or in the appliance. The appliance is preferably thermally stabilised using an internal temperature sensor and heater; in particular, the fluid reservoir 306 is preferably insulated and thermally stabilised to create controlled temperature conditions in the fluid flow, and so is provided with for example a temperature sensor 320 and a heater block 322. A control means 340 detects outputs from the sensors and controls the heaters to maintain a pre-set temperature or temperature profile in the wells, and controls flow of medium according to a pre-set programme.

FIG. 13 shows a schematic diagram of the system of the invention, comprising the device as in any of the previously described embodiments that provide fluid flow through the device, and an appliance for use with the device.

The appliance comprises an insulating enclosure 402 that contains the device and either the whole or other parts of the flow system. The insulating enclosure is openable to insert the device 400 and may comprise more than one insulated compartment whose temperatures are either jointly or separately controlled by heat exchange means 322. The device 400 is mounted on a heat exchange block 52 as before, equipped with a heater 318 and a temperature sensor 316, though the heat exchange block might be capable of cooling also, so comprising a peltier device coupled to a heat sink, or a block comprising channels for circulating cooled fluid to and from a refrigeration unit integrated as part of the system (not shown). The flow system comprises one or more reservoirs for medium, 404, 406, a pump 308, inlet flow line 408 and outlet flow line 410 with fluidic connections to the ports of the device, and a waste reservoir 412. The pump may be on the inlet side of the device or on the outlet side as shown dotted at 414.

A preferred embodiment of the flow system for the system of the invention is shown in FIG. 13. In common circumstances there is a need to change a first medium to a second during the course of culture. The flow system in FIG. 13 allows this to be done without need for valves to select the media, and with only a single pump. Reservoir 404 is filled with the first medium through port 420 and valve 422; reservoir 406 is filled with the second medium through port 424 and valve 426. Reservoir 406 is vented through a breather 430 and waste reservoir 412 vented through a breather 432. Flow channel 428 is optionally adapted to have a capillary stop at its exit to reservoir 406, and is filled with medium 1 during the filling of reservoir 404. The capillary stop means that in the absence of flow pressure medium 1 does not enter reservoir 406. In some embodiments a valve may be provided to close the channel 428. The pump 308 then draws medium from the reservoirs and flows them through the device. A debubbler 434 is optionally provided to capture bubbles from the system. Alternatively, a valve arrangement may be provided in the inlet flow line 408 to prime the system and remove bubbles before flow of medium is started. As reservoir 404 empties, the contents of reservoir 406 enter it and in turn are drawn through the pump and flow to the device. The reservoirs are preferably made of high aspect ratio to control mixing during the flow.

The insulating housing 402 may also be gas-tight, so as to contain a gaseous atmosphere for gas exchange with the medium in the reservoirs, the device or both. The reservoirs may therefore be provided with breathers to assist this process, the breathers being made for example from a porous hydrophobic polymer. The breathers may alternatively vent to the external atmosphere. The valves 422 and 426 are in preferred embodiments replaced by manual sealing caps in the ports 420, 424, arranged to be sealable without trapping air in the reservoirs.

The system of FIG. 13 is provided with control means 340 that acts to monitor and control the temperature in the various parts of the system, to control the flow and monitor the various sensors that are provided as part of the device or elsewhere in the flow system.

Other configurations of the device, appliance and flow system are envisaged for use in the system of the invention. For example, a flow system as known in the art, where a number of reservoirs are connected to a common flow line and flow controlled by valves associated with each, or separate pumping means associated with each, might also be used. Pumping means for the system include displacement pumps, pressurization of the medium either by gas pressure within the reservoirs or by deformation of the reservoir walls by mechanical actuation or external fluid pressure, or any other means known in the art.

The reservoirs, pump means and other flow components may be integrated onto the device itself, or the device and all or part of the flow system might be integrated into a subassembly which itself interfits with the transport module or appliance and remaining parts of the system.

FIG. 14 shows a schematic diagram of an embodiment of a system according to FIG. 13, with common parts numbered in common. The system 450 comprises a device 400 mounted inside a transport module or appliance (not shown), the appliance having an insulating housing (not shown) which houses the components of the flow system, control means and a power supply (not shown). The device and the reservoirs are shown on opposite sides of the appliance—this is purely schematic, and they could be in any practical disposition, but the system is intended in shipping to operate in any orientation and so the arrangement in FIG. 14 is practically relevant. The reservoirs in a practical embodiment are closed by stoppers 454, 456, which displace liquid towards the breather on closing the reservoir. In some embodiments the components of the system are mounted or formed in a solid block of material 452, preferably heat-conducting, which maintains uniform temperature throughout the system. Alternatively, the system is sufficiently well insulated that the inside is effectively of uniform temperature while operating. The appliance is closed by one or more lids 462, 464, which are designed to close and optionally to seal, held by clips and optionally hinged. In a preferred embodiment the system comprises a gas space in fluid communication with the device, that acts as a gas reservoir, and one or more gas inlets 470, optionally valved, are provided to flush and fill the gas space from an external gas supply 472 before transport. In FIG. 14 this space 468 is shown as being inside the lid 462, but it may be located elsewhere. The space may be pressurised or at atmospheric pressure. Alternatively a gas reservoir may be provided separately which is closed from the rest of the system and acts to supply gas to the device through specific gas lines and channels, in some embodiments formed on the device itself.

In a further embodiment the device additionally comprises a memory such as a microchip-based, or magnetic strip-based, memory system that allows data about the device and its contents to be recorded, read, stored, transported along with the device. In a preferred embodiment the memory and associated control circuitry is mounted on or within the device, together with a power source where needed. The memory system may be connected to other systems off the device by means of electrical contacts, wireless or optical communication, or it may be recorded and read magnetically. In a preferred embodiment the memory system contains information about the identity, history, contents, next actions and operational information concerning the objects and media in use on the device.

The memory system might comprise a device control system which acts to control functions on the device either independently of, or together with, the control system of the appliance, for example to indicate the status of objects in particular wells on the device and to prompt or prevent intervention by a user in the case of the whole device, objects in all, or in just some of the wells.

In a preferred embodiment the device is operable in conjunction with a further control means associated with observation of the objects on the device, for example by microscopy, in which the microscope control means is able to read from or write to the memory on the device, details of the objects in the wells of the device, media conditions, experimental observations and instructions for next actions either by the system comprising the device and the appliance, by a future experimenter, or both. In preferred embodiments the memory system of the device interacts with a laboratory information system to control the use and operation of the device and/or the appliance so as to track the use, record the conditions, or ensure compliance with record keeping or other regulatory activities.

The above embodiments require the mounting onto or within the device of an electronic system, examples of which are known in the art, and the provision of electrical contacts as disclosed for several of the embodiments above. Alternatively, wireless communication may be made between the device, the applicant or another off-device system. In either case the design required to mount the memory system on or within the device is standard and known in the art.

In a further embodiment the appliance additionally comprises one or more of the following:

data logging means that records data from the sensors of the system, such as the temperature, pH, dissolved oxygen or other sensors as described above associated with conditions in the medium to which the embryos are exposed;

sensors elsewhere in the system, such as internal and external temperature sensors which measure the correct functioning of the system and the environmental conditions in which it is located;

accelerometers and attitude sensors which might be provided to detect motion, shock or untoward events;

communication means that allows communication between the appliance and a remote system, such as a mobile telephony interface or a wireless data interface;

GPS position monitoring means;

which together with the control means of the appliance can act to monitor or control the operation of the appliance and the device, log its position and report status and positional information to a remote station.

It is useful in the case of loss or delay in transport to be able to locate the transport system of the invention and optionally to receive information on its status and the status of the objects within it. The above features allow this to be done.

A system is provided for transporting embryos comprising a device having wells for the embryos, the wells being closed by a lid, and a transport module or appliance as described above acting to:

control the temperature of the embryos,
optionally control the composition of the medium in the wells,
optionally provide a controlled gaseous environment,
log conditions on the device,
log conditions in the rest of the appliance,
optionally log condition external to the appliance,
and in certain embodiments the appliance comprises communication means which allow communication between the appliance and external apparatus, such as GPS position logger, a mobile telephony interface, a wireless data interface, which can act to monitor or control the operation of the appliance and the device, or log its position, and transmit data to a remote location.

It is an object of the invention to provide an apparatus and method for transporting a payload at a controlled temperature, in which drawbacks in the apparatus of the prior art are overcome. Such drawbacks include: poor temperature regulation; short endurance before temperature drifts out of specified range; large size and/or weight to achieve endurance of the order of 4 days or more; tendency of cool transport apparatus, intended to maintain temperatures close to 0 C, to freeze the sample when this is first loaded into the apparatus and compromises to the performance of the apparatus introduced to counteract this; and lack of ability of warm transport apparatus, intended to maintain temperatures above mean ambient, to resist over-temperature for extended periods. Prior art apparatus all suffer from at least one of the above problems. Mean ambient temperature is defined in the following as a mean temperature in the range approximately 10-25 C.

Nagle U.S. Pat. No. 6,020,575 discloses apparatus intended for shipping at above mean ambient temperature, having an outer insulation layer defining an inner space, with an electric heater and a eutectic material (or “Phase Change Material”, PCM) together closely adjacent in the inner space, the eutectic material intended to assist in the heating action.

Rix U.S. Pat. No. 6,822,198 discloses a transport apparatus comprising an insulating housing enclosing an inner electric heater and a cooling pack. The position of the cooling pack relative to the heater is not disclosed, and there is no insulation between the heater and the cooling pack. This apparatus has no feature to prevent contact between the cooling pack and so potentially suffers from uncontrolled heating of the cool pack by the heater, and so in use will have variable and potentially short endurance; also, uncontrolled temperature gradients will exist within the chamber between the heater and cooling pack.

Nadeur WO03/101861 discloses a shipping device including a body comprising PCM surrounding and in contact with a payload, the PCM having a melting point Tc substantially the same as the storage temperature for the payload. This device will keep the temperature stable once the PCM has reached Tc, but in order to freeze the PCM it needs to be cooled some way below Tc. In order to warm the PCM to Tc, it needs to be conditioned, i.e. warmed, which takes time, is prone to error, and owing to the extended range of melting which many PCM have, wastes a considerable portion of the cooling capability of the PCM. Especially for apparatus operating close to 0 C, there is a danger of an aqueous payload freezing, which is to be avoided for biological samples.

No transport apparatus is known in the prior art that combines high capacity coolant with the ability to use a conventional freezer at −15 C to −20 C to freeze the coolant, in a design which will substantially prevent a payload cooling below 0 C, while providing a interior temperature close to 0 C.

Temperature controlled transport apparatus operating at temperatures above mean ambient are known, for example to transport living biological samples at the temperature range 37-39 C. These apparatus usually rely on insulation and an inner heating means, for example pre-heated PCM or an electric heater powered by a battery pack, and have an endurance that is limited by the capacity of the battery or PCM and by the insulation. The apparatus of the prior art adapted for small scale transport of biological materials have no refrigeration capability however, and so are liable to overheating in high ambient temperatures, such as are likely to be encountered in the course of shipping in warm climates.

Over-temperature protection for temperature-sensitive goods is disclosed by H of et al. U.S. Pat. No. 4,425,998, which provides a layer of PCM in the form of a salt with a melting point Tc just below the sensitive temperature of the goods, surrounded by an insulating outer housing. The arrangement of H of et al. is not suitable for protection of a heated payload, however, as heat flux from the payload (which is above Tc) will tend to melt the protection salt.

The further the melting point of the salt is below the operating temperature, and the better is the external insulation, so the better is the thermal protection, but the greater is the tendency of the salt to be melted by the heated payload. The present invention differs from the design of H of et al. by providing an inner insulation layer and by selecting advantageous combinations of the insulation and PCM parameters.

The invention provides an apparatus for transporting a payload at a controlled temperature, comprising an outer housing, outer insulation region, a heat sink region comprising a heat sink such as a heat absorbing material, in some embodiments a heat sink component such as a cold body, which may be pre-cooled before introduction into the apparatus; an inner insulation region and a heated payload. The apparatus can be adapted to operate at any required temperature in the range from below zero to significantly above mean ambient temperature.

In a first preferred embodiment the apparatus is adapted for use at above mean ambient temperature, such as in the range 37-41 C, for example for incubation and transport of cellular entities such as cells in culture, embryos or oocytes; in this embodiment the heat sink is preferably in the form of a heat absorbing material and acts to protect the apparatus against over-temperatures resulting from prolonged exposure to high ambient temperature.

In a second preferred embodiment the apparatus is adapted for use at below mean ambient temperature, such as in the range 0-10 C, for example for transport of tissue samples, organs, blood or blood products or temperature-sensitive pharmaceuticals or other chemicals; in this configuration the heat sink is advantageously in the form of a heat absorbing material in one or more containers that are reversibly removable from the apparatus, and may be cooled before being introduced to the apparatus.

In either of the above embodiments the heat absorbing material is preferably a phase change (PCM) or eutectic material that has a transition temperature lower than the desired control temperature of the payload. In the higher temperature case, the PCM preferably has a transition temperature in the range 10 C-1 C, more preferably in the range 5 C-2 C; i.e. allowing a small margin of temperature protection below the desired operating temperature. In the lower temperature embodiment slight over-temperature is most likely less important, so the PCM preferably has a transition temperature in the range 10 C-0 C below, more preferably in the range 4 C-1 C below the desired operating temperature. A particularly preferred embodiment for use in the range 1 C to 4 C uses a water-based heat absorbent material with a transition temperature close to 0 C.

FIG. 15 shows a diagrammatic cross section of a first embodiment of the apparatus. The apparatus 500 comprises a base 502 and a lid 504, which is preferably a close fit to the base and may be held in place or held closed by closure means (not shown). The apparatus comprises an outer housing 506, comprising an outer insulation region 508 and a heat sink region 510 comprising a heat absorbing material. In preferred embodiments the heat absorbing material comprises a phase change material (PCM) chosen to have a mean transition temperature below the desired operating temperature of the payload. The apparatus further comprises an inner insulating region 512, disposed between the heat sink region and the payload space 514. The payload space is opened for access by opening the lid 504, and in preferred embodiments comprises a payload unit 516 which is reversibly removable from the apparatus.

The payload unit comprises an inner housing 518, which holds a payload 520, a heater unit 522 which heats the payload, a control means 524 and a power supply (for example batteries) 526. The control means measures the temperature of the payload by means of a temperature sensor 528. In some embodiments the sensor 528 is mounted on the payload itself; in other embodiments the payload is housed in a payload container (not shown in FIG. 15), the heater heats the payload container, and the temperature sensor may be mounted either on the payload container or the payload itself. The arrangement of the heater and payload in FIG. 15 is diagrammatic and other arrangements are possible—for example the heater may be located within the payload, or may be distributed around it. Preferably the control means also reads the ambient temperature by means of a sensor 530.

In preferred embodiments one or both of the insulating regions comprise one or more vacuum insulation panels (VIPs). The outer housing may additionally comprise insulating and/or shock absorbing material, for example expanded polystyrene (EPS).

In use the heater controls the payload temperature against heat flux to ambient. When the ambient is below the control temperature heat is lost through the inner insulation, the heat absorbing material and the outer insulation. The heat absorbing material is chosen to have a higher heat capacity than the insulation and acts to buffer the heat flux to/from ambient by absorbing and giving out heat. In the case that the heat absorbing material is a PCM, the PCM acts as a thermal reservoir at or near the transition temperature. The operation of the apparatus is illustrated by the example that the control temperature of the payload is 38 C, suitable for culture of embryos. The particular advantage of the apparatus in FIG. 15 is that the heat absorbing material acts to protect against high ambient temperature. A PCM with a transition temperature Tc in the range 30-35 C is preferably used. When the ambient temperature is significantly below Tc the PCM is frozen and acts as a conductive link between the inner and outer insulation regions. The rate of heat loss to the ambient depends primarily on the sum of the thermal resistances of the insulation regions. When the ambient temperature rises above Tc, heat flows from ambient to the PCM and this gradually melts, absorbing heat, so substantially preventing heat flux to the payload. Finally the PCM has melted entirely and then acts once again as effectively a conductive link, and over-temperature protection is exhausted. At this point the payload temperature will begin to rise. Once the ambient temperature falls below Tc, the PCM will gradually freeze and a degree of over-temperature protection will be regained.

The endurance at ambient temperatures above Tc depends on the heat capacity of the heat absorbing region and the thermal resistance of the outer insulating region, and these are chosen to give an advantageous compromise between protection and size and weight of the apparatus. The sum of the thermal resistances of the inner and outer insulating regions determines the power requirement of the heater and the endurance of the apparatus for given battery capacity at low ambient temperatures. In the case that the heat absorbing material is a PCM, for over-temperature protection to work the PCM should be substantially frozen when the apparatus is in a normal temperature ambient. In preferred embodiments of an apparatus operating at 38 C, with a PCM with Tc around 35 C, the outer insulating region preferably has a lower thermal resistance than the inner region. This means that the PCM is poised closer to mean ambient temperature than 38 C, so keeping it frozen. However, the less the outer-thermal insulation, the greater the heat capacity of PCM that is needed to maintain protection against over-temperature. For preferred embodiments in which the inner and the outer insulation comprises VIPs, the tradeoff is between thickness of VIP and thickness (and mass) of PCM. In a typical embodiment of the apparatus, adapted to operate at a control temperature in the range 37-40 C, preferred ratios of thickness of the inner to outer insulation are between 1:1 and 4:1. For embodiments designed to operate at control temperatures closer to mean ambient temperature, the optimum ratio will be different: Tc of the PCM will be lower, and a greater proportion of the total insulation is advantageously placed outside the PCM to slow heat conduction to the PCM in over-temperature conditions. The ratio of outer to inner insulation is chosen according to the design requirements of the apparatus.

In a typical embodiment of the apparatus, adapted to operate at a control temperature in the range 37-40 C, using VIPs of thermal conductivity 0.0042 W/mK (Vaq-VIP from Va-Q-Tec GmbH, Wurzburg, Germany) and PCM with Tc 35 C and latent heat capacity 99 kJ/litre=500 kJ/m2 for 5 mm thick panels (Rubitherm RT35 in fibreboard form, Rubitherm GmbH, Hamburg, Germany), the ratio of thickness of the inner to outer insulation may be chosen to be between around 1:1 and around 4:1. Examples of preferred embodiments are given, but no limitation to these is to be understood. Preferred embodiments using these materials have outer VIP in the range 5-15 mm thick, PCM layers in the range 5-10 mm thick and inner VIP layers in the range 1 to 4 times the thickness of the outer VIP. A preferred embodiment has an outer insulation VIP approximately 5 mm thick, a PCM layer 8 mm thick and an inner VIP approximately 20 mm thick. This combination will give over-temperature protection for a transport appliance with a control temperature of 38 C against 50 C ambient for around 8 hr. A further preferred embodiment has an outer insulation VIP approximately 8 mm thick, a PCM layer 5 mm thick and an inner VIP approximately 17 mm thick. This combination will give also over-temperature protection for an apparatus with a control temperature of 38 C against 50 C ambient for around 8 hr.

In the embodiment in FIG. 15 the heat sink region is shown as extending substantially around the apparatus and heat absorbing material in the heat sink region is evenly distributed between the outer and inner insulating regions. In this embodiment if ambient heat energy arrives at the outer housing preferentially on one face, for example from sunlight, heat will flow from that face primarily to the adjacent heat absorbing material in the heat sink region. A certain amount of heat will be conducted from the locally heated heat absorbing material to material on the adjoining faces, but this will be limited by the thermal conductivity of the heat absorbing region, which will be limited if it is thin or the heat absorbing material is provided in the form of discrete panels. Therefore in an alternative embodiment the heat absorbing material, for example PCM in panel form, is contacted by a layer of conductive material, for example metal, that acts to conduct heat away from the region of high heat incidence. In a further preferred embodiment the heat absorbing material is provided in localized regions between the outer and inner insulation, and a layer of thermally conductive material, for example metal, is provided substantially surrounding the inside of the inner insulation, which acts to conduct heat from the inside of the outer insulation to the regions of heat absorbing material. In preferred embodiments of this type, the total amount of heat absorbing material may be reduced relative to embodiments where the heat absorbing material is provided in-situ at each face of the apparatus to absorb heat arriving at that face.

The apparatus may be of any desired shape (though is most easily fabricated with rectangular faces) and may be fabricated from a variety of materials and in a variety of ways as known in the art. The insulation regions are preferably formed from VIPs, either discrete panels for each face of the apparatus or one or more continuous panels formed to fit the outer housing. Examples of suppliers of suitable panels are ‘Va-Q-VIP’ from Va-Q-Tec GmbH, Wurzburg, Germany; ‘VacuPanel’ from Technautics Inc., Costa Mesa Calif., USA; VIP (unbranded) from ThermoSafe Inc., USA. The VIPs are preferably protected by thin protective layers or liners (not shown in FIG. 15) formed e.g. from puncture resistant plastic. Examples of suitable heat absorbing materials for use in the heat sink region are sheet-form PCM, for example ‘Rubitherm’ from Rubitherm GmbH, Hamburg, Germany, which are available in a variety of thicknesses and Tc values and may be assembled simply in close alignment with the VIP panels inside a ruggedised transportation housing. The heaters, temperature sensors control means and power supply are of types known in the art. The control means preferably comprises a microprocessor and programming means to provide an operating program to control operation of the apparatus, for example to control the heater(s), charging of the batteries, log readings from the temperature and other sensors and provide input and output functions.

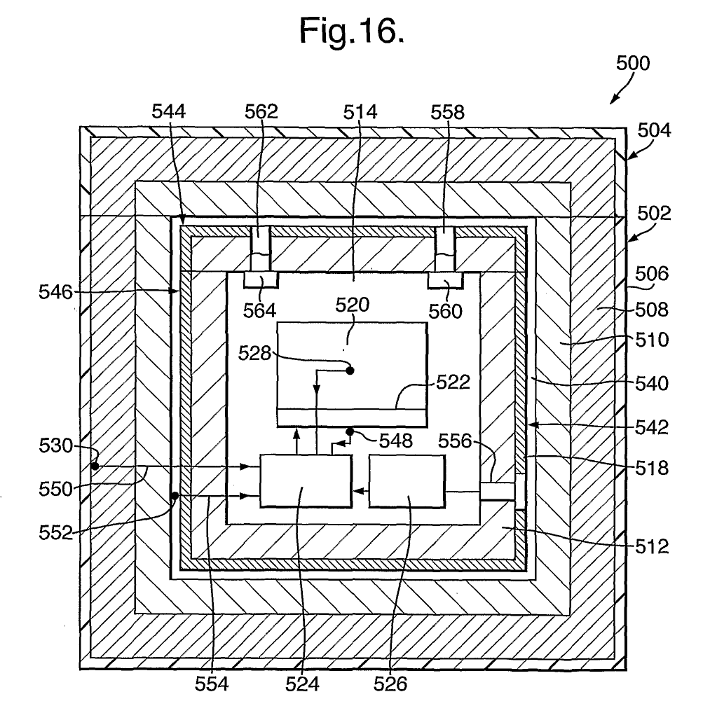

FIG. 16 shows a further embodiment of a apparatus 500 comprising a body 502 and a reversibly openable lid 504. The apparatus comprises an outer housing 506, outer insulating region 508 and heat sink region 510 comprising heat absorbing material, that together define an inner insulated space 540. An inner insulated unit 542 is in preferred embodiments reversibly removable from the apparatus and comprises a housing 518 and an inner insulation region 512, which define a payload space 514. The inner insulated unit further houses a payload 520 and heater 522, control means 524 and power supply 526. The control means reads temperature sensor 528 which indicates the temperature of the payload, or in embodiments in which the payload is housed in a further container (not shown), optionally the temperature of the container. In preferred embodiments the control means reads an ambient temperature sensor 530. The control means optionally also reads additional temperature sensors 548, which measures the temperature of the heater, and/or 552, which measures the temperature of the heat sink region, and/or further temperature sensors (not shown) that read the temperature of the interior of the inner insulating region. In the embodiment in FIG. 16, the leads 550 and 554 from the sensors 530 and 552 pass through the insulating and heat sink regions, and into the inner insulated unit 542 in such a way that the lead can be extended or unplugged to remove the unit 542. In some embodiments the sensor 528 on the payload itself is omitted and the reading from sensor 548 used instead to control the payload temperature.

In a preferred embodiment the inner insulated housing 542 is gas-tight, so allowing a different gas atmosphere to be maintained in the space 514 from in the rest of the apparatus. This is advantageous for example if the payload contains cells in culture, embryos, oocytes etc., in media which require a CO2 atmosphere for pH control. In this case, the lid 544 of the inner insulated housing preferably has a gasket or O-ring pressure-tight seal to the base of the inner housing. Gas inlet 558 closed by valve 560, and gas outlet 562 closed by valve 564 are provided to introduce a gas atmosphere into the inner housing 542. Power line connector 556 is adapted to be gas-tight also.

In the embodiment in FIGS. 15 and 16 the control means and power supply are shown as being inside the inner insulation. It will be understood that embodiments in which either or both are elsewhere in the apparatus are included in the invention. In a preferred embodiment both are located either between the outer insulation and the inner insulation, or between the outer housing 506 and the outer insulation.

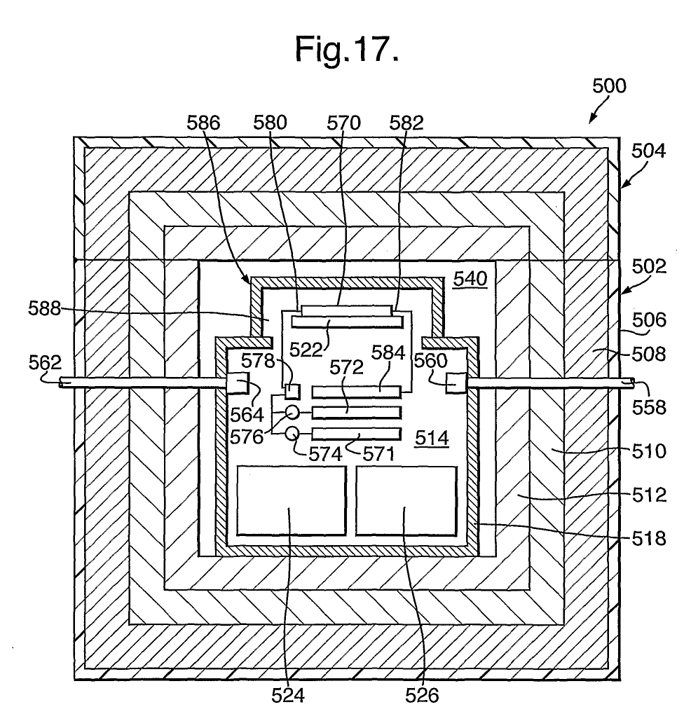

FIG. 17 shows a further embodiment adapted to house and transport a payload in the form of a fluidic device 570, such as a microfluidic device adapted to house and culture cellular entities such as cells, embryos or oocytes, in a controlled temperature and gas environment. Apparatus 500 again comprises a body 502, lid 504, outer housing 506, outer insulation region 508, heat sink region 510 and inner insulation region 512, together defining an inner space 540. An inner housing 518 is gas-tight in this embodiment, defining a payload space 514 that can contain a gas atmosphere different from that in space 540 or ambient.

Gas inlet 558, inlet valve 560, outlet 562 and outlet valve 564 are provided to allow gas to be flowed into the space from outside the apparatus once the apparatus is closed. In a preferred embodiment, housing 518 is closed by a gas-tight lid 586. In some embodiments lid 586 defines an upper payload space 522 which may be separate from the main payload space 514. In FIG. 17 the two spaces are shown as being open to each other. Control means 524 and power supply 526 are provided as before, with temperature sensors as in any previous embodiment (not shown).

The embodiment in FIG. 17 has a fluidic circuit adapted to enable flow of liquid media through the device 570, comprising fluidic reservoirs 571, 572, each with a control valve 574, 576; a pump 578, an inlet line 580 to the device and an outlet line 582 leading to a waste reservoir 584.

In this and previous embodiments the heating means is preferably an electric heater. In an alternative embodiment the heating means comprises a fluidic heat conducting means which acts to heat the payload from a heat source, for example an electric heater, elsewhere in the apparatus, for example by means of fluid flowing through heating channels in the body of a microfluidic device 570.

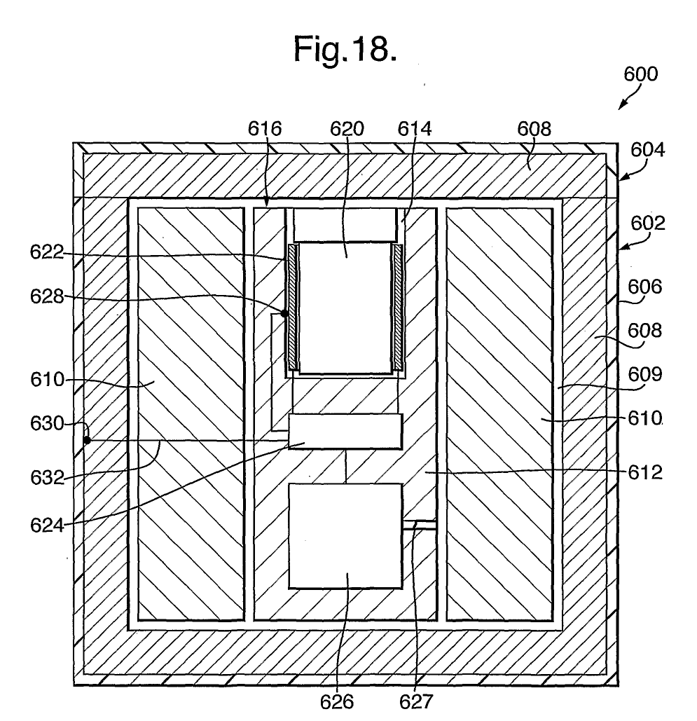

FIG. 18 shows a further embodiment, adapted to control the temperature of the payload space at or below mean ambient temperatures. The apparatus 600, comprising a body 602 and lid 604, comprises an outer housing 606 and an outer insulation region 608, together defining an inner space 609, and within that space a heat sinking region for receiving one or more heat sink components 610, reversibly removable from the apparatus. The apparatus further comprises an inner unit 616, which in preferred embodiments is also removeable from the apparatus, comprising an inner insulation region 612, a payload space 614, and which may comprise a further housing component (not shown) external to the inner insulation region. In FIG. 18 the payload 620 comprises a lidded payload container and an inner payload content (not shown). This embodiment is adapted for transport of biological materials such as tissue samples, biopsies, body fluids and the like which need a secondary containment around the primary sample container.

The payload container shown in FIG. 18 is cylindrical—though any form of payload or payload container is within the scope of the invention. At least a region within the payload space is heated by a heater 622, in a preferred embodiment disposed partially or substantially around the payload, for example in a cylindrical configuration to house closely the cylindrical payload container in FIG. 18. The heater is controlled by the control means 624 in response to sensor input from a temperature sensor 628, shown adjacent the heater, but which may be located elsewhere, for example in close proximity to the payload container, or within the payload container or closely adjacent to, mounted on or within the payload. Further temperature sensors, for example an ambient temperature sensor 630, may be provided and are optionally read by the control means. In an alternative embodiment, the ambient sensor 630 is an autonomous sensor, such as an ‘i-button’ from Maxim Inc. or a ‘heat button’ from Heatwatch Inc., which has the advantage that no connection 632 is needed between the inner unit 616 and the sensor. Power supply 626 is provided within the unit 616, which may be connected to line power when the unit 616 is removed from the apparatus using line power connection 627.

In a preferred embodiment, adapted for use in the temperature range 0-10 C, for application for example in transport of tissue samples, the heat sink components 610 comprise a water-based coolant. The components may take the form of bottles, adapted to fit into the heat sink region in space 609, or in alternative embodiment may be conventional gel packs in flexible packaging and frozen in a shape that allows them to fit into the heat sink region.

In use in preferred embodiments the heat sink components are frozen in a conventional freezer and may be placed in the insulated housing straight away from the freezer. The inner unit, comprising the payload, heater, control means and power supply, may have the batteries charged while outside the apparatus, and pre-set using controls on the inner unit to the desired temperature, then inserted into the apparatus adjacent to the heat sink components. The sensor 628 detects the fall in temperature resulting from conduction through the inner insulating region 612 to the heat sinks, and the control means heats the payload space to maintain the desired temperature against cooling from the heat sinks. The lid 604 is fitted, and the apparatus may now be shipped. Once the heat sinks reach around 0 C the temperature remains nearly constant—the heater then runs to maintain the differential between the control temperature and 0 C. For low control temperatures, e.g. 2 C as appropriate for tissue samples, only very low power is needed to do this, as a result of the inner insulating region. Prior art transport systems which do not have such an inner insulating region have a much higher power requirement, with consequent short endurance from a given battery capacity, and rapid loss of cooling capacity. Outer insulation 608 serves primarily to insulate the coolant from melting; the inner insulation controls the temperature gradient between the payload and the heat sinks 610.

A great advantage of this embodiment is that a sample can be kept close to 0 C without the danger of freezing and consequent degradation of the sample. Also, compared with transport apparatus of the prior art in which payload temperatures are kept above 0 C by buffering with water at 4 C or using PCM with transition temperature above 0 C, the apparatus of the invention has a much longer endurance for a given size and weight. The water used for buffering contributes little cooling capacity per unit volume and mass; the PCMs with transition temperatures at say 4-6 C have both lower specific latent heat and lower density, so having a latent heat per unit volume as low as half that of water. Additionally, pre-conditioning (partial thawing) of the coolant, necessary even when using PCM with Tc above 0 C in prior art non-heated transport apparatus, is not necessary, so avoiding a significant source of potential failure in the transport protocol.

Control temperatures significantly above 0 C may be achieved with the embodiment above, at the cost of increased power required for the heater. Preferred embodiments for operation at significantly above 0 C may have more insulating inner insulation regions 612. In preferred embodiments a PCM is used in the heat sink components that has a Tc value within a limited temperature range at or below the desired control temperature, in order to minimize the battery capacity needed for a given shipping endurance. For example, in a preferred embodiment adapted to run in the temperature range 8-15 C, a phase change material with Tc at 4 C-8 C may be used instead of ice, and for the range 10 C and above, a phase change material with Tc in the range 5 C-10 C may be used. In general in preferred embodiments a PCM is used that has a Tc around 0 C-20 C below the control temperature, in more preferred embodiments 1 C-10 C and inmost preferred embodiments 1 C-5 C.

In preferred embodiments the outer insulation comprises at least one VIP panel, and in more preferred embodiments one VIP panel for each face of the apparatus. The insulating properties of the VIP are chosen with regard to the intended endurance of the shipper in given ambient conditions. In some embodiments VIP panels are use also for the inner insulation region. In preferred embodiments the requirements for the inner insulation are less strenuous than those for the outer insulation and so other insulation materials, for example structural polymer foam, may be used. The inner unit may be housed in a structural housing (not shown) if required.

An experimental apparatus of the embodiment of FIG. 18 was constructed with an outer housing comprising six VIP panels of thermal conductivity 0.0042 W/mK (Va-Q-VIP from Va-Q-Tec, GmbH) 230×230×20 mm thick, and 2.7 kg of ice/gel packs with Tc=0 C and latent heat capacity 330 kJ/kg, placed in a heat sink region defined by four plastic containers 210×160×40 mm. The inner insulation was a polyurethane foam block, thermal conductivity approximately 0.03 W/mK, 110×110×210 mm, with a cylindrical payload container 70 mm diameter located axially within it, giving a minimum inner insulation region thickness of 20 mm.

A thin 50 W sheet-form heater was mounted on the inside of the inner insulation, around the payload container 620, and a heater control means set to a control temperature of 1 C was connected with a temperature sensor adjacent the heater as shown as 628. An ‘i-button’ temperature logger was placed on the inside of the payload container. The mean ambient temperature was around 20 C. The frozen ice packs were placed in the heat sink region at −18 C. The temperature inside the payload container had reached 1 C in around 10 hr and remained within 0.25 C of 1 C for an endurance of greater than 7 days (at which time the test was terminated). Total energy consumption over 7 days was 2.5 kJ (mean power 4 mW). For comparison, in experiments using the same housing, outer and inner insulation but without active heating, using PCM with Tc 4-6 C and specific heat capacity 2.4 kJ/kg and relative density 0.8 filling the containers, inserted into the apparatus at −18 C, the payload temperature fell below 0 C within 3 hours. Used without electric heating ice-based gel packs, with specific heat capacity of 4.2 kJ/kg, would be expected to cool the payload below 0 C in an even shorter time. Using the Tc=4-6 C PCM the payload temperature rapidly reached 4 C and drifted steadily upwards to reach 8 C after 4.5 days, beyond which the PCM had melted completely and the temperature rose rapidly. The apparatus of the invention had better short term resistance to freezing, better temperature regulation, and much longer endurance for a given size and weight than the comparable apparatus without the configuration of the invention.

Alternative embodiments to that in FIG. 18 are within the scope of the invention. For example, additional heat sink components may be located above and/or below the inner unit 616, and the inner insulation may additionally extend above the payload container 620.

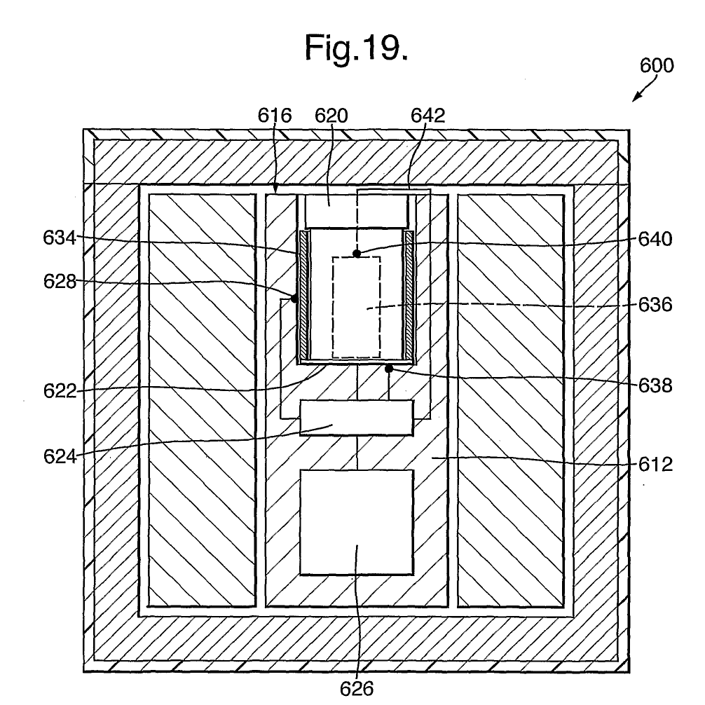

FIG. 19 shows a further preferred embodiment, in which an apparatus 600 has parts in common with that in the embodiment in FIG. 18. An inner unit 616 comprises inner insulation region 612, payload container 620, heater 622 and control means 624 as before. Here the heater is disposed primarily at the base of the payload space and heat is conducted round the payload space by one or more conductive components 634, for example a metal cylinder in good thermal contact with the heater. Temperature of the conductive component(s) may be controlled by temperature sensor(s) 628 in contact with the components, and optionally additional sensor(s) 638 in contact with the heater and 640 in contact with a payload 636 within the payload container as required.

In an alternative embodiment (not shown) the heater or conductive component(s) may be shaped to interfit with the payload container or the payload itself to give good thermal contact between them. For example, the heater or conductor may be in the form of a rod onto which the container fits, so giving a radial heat flux from the centre of the payload container, outwards to the inner insulation and thence to the heat sinks.

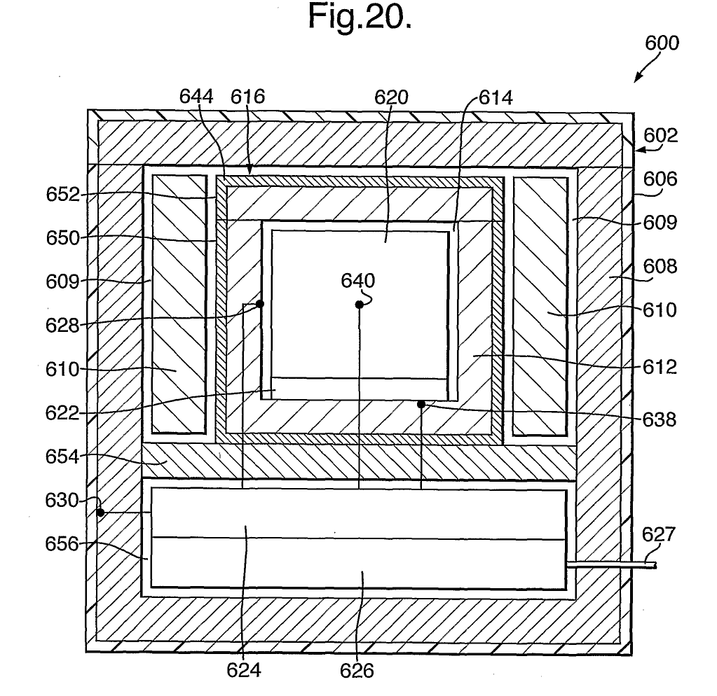

FIG. 20 shows a further preferred embodiment, adapted to contain a payload in a lidded inner payload space within the apparatus. The apparatus 600 comprises an outer housing 606, outer insulation region 608, an inner unit 616 in this embodiment formed as part of the structure of the apparatus, and an inner partition 654, together defining one or more heat sink regions 609, that receive reversibly removable heat sink components 610, and a space 656 which contains the control means 624 and power supply 626, connected at times by a power line connection 627 through the body of the apparatus. The inner unit 616 comprises an (optional) inner housing 644 and inner insulation region 612, and has a base 650 and reversibly openable lid 652, which define a payload space 614. The payload 620 is shown as being a container of any suitable form to fit the space 614. Heater 622 is located in the payload space. Temperature sensor 628 may be provided to sense the temperature of the payload space, 638 to sense the temperature of the heater and 640 to sense the temperature of the payload container or the payload itself.

Temperature sensor 630 is optionally provided to read ambient temperature. Control means 624 and power supply 626 are separated from the payload space by inner insulation 610 and from the heat sink components 610 by the partition 654 which in preferred embodiments is itself insulating to prevent heat from the control means and power supply reaching the heat ink components. The location of the power supply outside the inner insulation in this embodiment is advantageous where the power supply dissipates more heat, especially while charging the batteries, than can conveniently be lost through the inner insulation. In some embodiments parts of the power supply (such as power transistors or ICs) might be arranged to be in good thermal contact with the outer housing to allow dissipation while charging batteries.

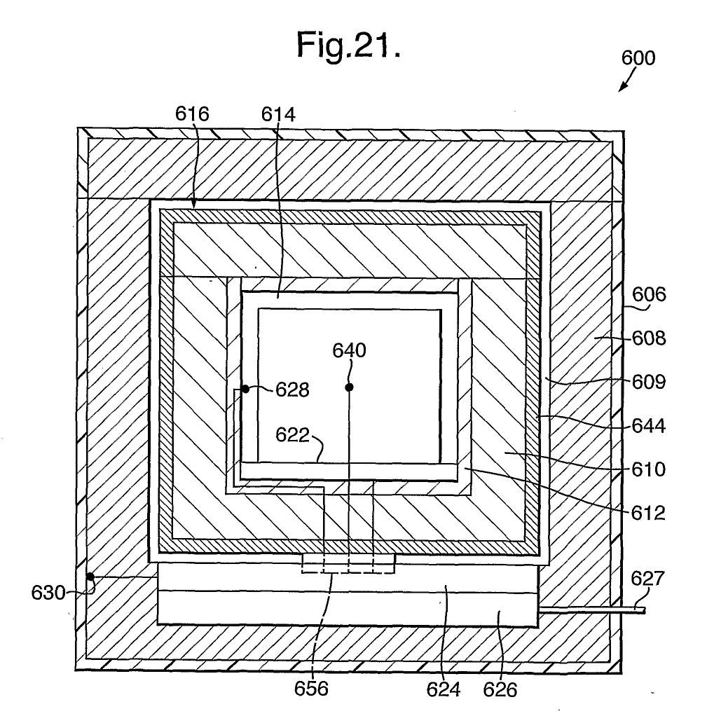

In the embodiments in FIGS. 18-20 the heat sink components have been shown as separate from the remainder of the apparatus, allowing easy cooling in a freezer. In an alternative embodiment the heat sink components are formed as an integral part of the apparatus, preferably of an inner unit which is reversibly removable from the apparatus, so allowing the complete unit to be removed and cooled. The unit may then be replaced en bloc in the apparatus. The apparatus in FIG. 21 has common parts with previous embodiments, numbered in common. In this embodiment the heat sink receiving space 609 is common with the space in which the removable inner unit 616 is received. Unit 616 comprises an outer housing 644, heat sink region 610, which in preferred embodiments comprises PCM, inner insulation region 612 and defines an inner payload space 614. The unit 616 has a lid which gives access to the payload space. The unit 616 is removed before use and cooled, to bring heat absorbing material 610 below its Tc. In a preferred embodiment unit 616 connects to the apparatus by means of a plug connection 656, for example at the base of the unit.

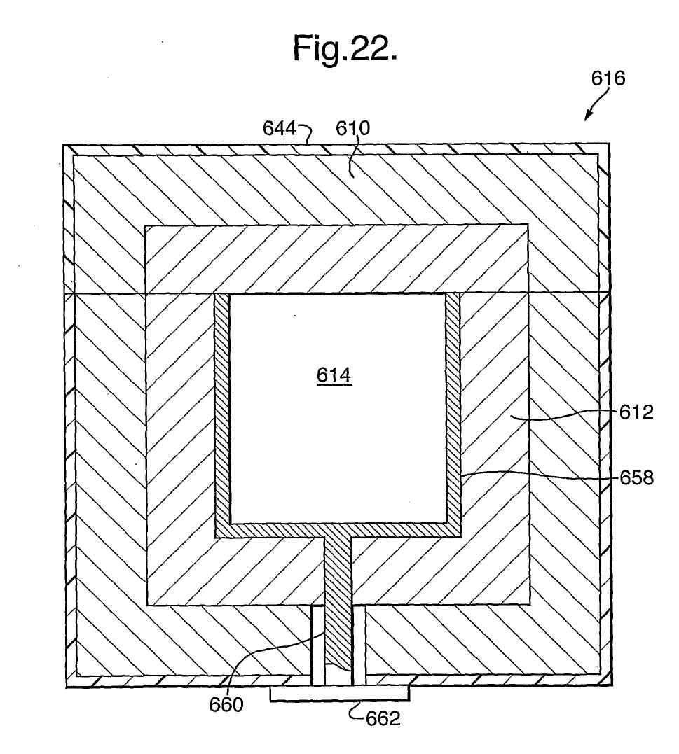

FIG. 22 shows an alternative embodiment, in which the heater is located as part of the apparatus, external to the unit 616. Unit 616 comprises a sample space 614, in thermal communication with a thermally conducting component 658, in a preferred embodiment adapted to achieve a substantially uniform temperature in the space 614, for example disposed around the perimeter of 614. The thermally conducting component is in thermal communication with the exterior of unit 616 via thermally conducting region 660, to a thermal contact means 662 which is brought into thermal contact with the heater 622 (not shown in FIG. 22) when unit 616 is inserted into the apparatus. In this way unit 616 can be a passive component without the need for electrical connections.

It will be understood that by using a PCM with a different Tc, an apparatus adapted for a different range of control temperatures can be constructed. For example, PCM at the following temperatures is known to be available: −4, −1, 0, 2-6, 3-9, 5, 7, 20-22, 24, 26-28, 29, 32, 33-38, 35-36, 44-45, 48, 58. Apparatus suitable for use at control temperatures in the range 0-20 C, preferably 1 C-5 C above Tc can be fabricated and achieve temperature control using electrical heating to raise the payload temperature above Tc. In each case, the presence of an inner insulation layer between the PCM and the heater is essential to give optimum performance.

It will be understood that in the embodiments above the control means may be of any kind known in the art. In preferred embodiments, the control can communicate with external devices to upload programs, download data, give status updates etc., by means known in the art including RF, IR, Bluetooth, USB or other cabled connection.

In a further embodiment the apparatus additionally comprises one or more of the following:

data logging means that records data from the sensors of the system, such as the temperature, pH, dissolved oxygen or other sensors as described above associated with conditions in the payload;

sensors elsewhere in the system, such as internal and external temperature sensors which measure the correct functioning of the system and the environmental conditions in which it is located;

accelerometers and attitude sensors which might be provided to detect motion, shock or untoward events;

communication means that allows communication between the appliance and a remote system, such as a mobile telephony interface or a wireless data interface;

GPS position monitoring means;

which together with the control means of the apparatus can act to monitor or control the operation of the apparatus and the device, log its position and report status and positional information to a remote station.

It is useful in the case of loss or delay in transport to be able to locate the apparatus of the invention and optionally to receive information on its status and the status of the objects within it. The above features allow this to be done.

1. A device for culturing or maturing cellular entities, the device comprising a substrate having one or more wells, said one or more wells being adapted to hold a cellular entity, and lid means releasably-secureable to the substrate to prevent entry or exit of the cellular entity, wherein the device further comprises a source of a fluid and fluid transport means to feed the fluid from the source to the one or more wells in use.

2. A device for transporting at least one cellular entity during culture or maturation, the device comprising a substrate having one or more wells, said one or more wells being adapted to hold a cellular entity in a fluid, lid means for preventing entry or exit of the cellular entity from the one or more wells and fluid transport means connecting the one or more wells to enable flow of fluid or diffusion of chemical species.

3. A device as claimed in claim 1 or 2 wherein the one or more wells are open to a major surface of the substrate.

4. A device as claimed in any preceding claim in which the fluid consists of a gas and the fluid transport means comprises a gas permeable element.

5. A device as claimed in claim 4 in which the substrate or lid means consists of or includes said gas permeable element.

6. A device as claimed in claim 4 or 5 in which the gas permeable element consists of a porous polymer.

7. A device as claimed in claim any preceding claim in which the wells are adapted to prevent physical contact between cellular entities in adjacent wells while allowing chemical transport between the wells.

8. A device as claimed in any preceding claim in which the one or more wells are tapered to locate the cellular entity at a given location in the or each well.

9. A device as claimed in any preceding claim in which fluidic pathways are provided between a plurality of wells.

10. A device according to any preceding claim wherein the fluid transport means comprises a material which controls diffusion and/or convection of the fluid between wells.

11. A device as claimed in claim 10 in which the material is a member of the group consisting of: —porous hydrophilic polymers; polymers permeable to species in the liquid phase; hydrogels; filter materials.

12. A device as claimed in any preceding claim including means for altering the composition of a liquid medium in the one or more wells with time.

13. A device as claimed in claim 12 in which the means for altering the composition comprises substance release means including a substance to be released into the one or more wells or the liquid medium.

14. A device according to claim 13 further comprising release control means to control the timing and/or rate of release of the substance into the well or medium.

15. A device as claimed in either claim 13 or 14 in which the substance release means is in the form of a body which is provided in said one or more wells before the lid means is secured.

16. A device as claimed in any of claims 13 to 15 in which the substance release means is in the form of one or more layers of material on a wall of said one or more wells.

17. A device as claimed in claim 16 in which the substance release means comprises a layer of a substance to be released covered wholly or partially by a layer of a control material.

18. A device as claimed in claim 13 in which the substance to be released is provided in a reservoir having a fluidic pathway to said one or more wells and the release control means includes a barrier in said fluidic pathway made from control material.

19. A device as claimed in any of claims 13 to 18 in which the release control means includes a control material which is soluble in a liquid medium, or which becomes permeable on contact with a liquid medium.

20. A device as claimed in claim 18 wherein the control material becomes permeable or breached by an electrochemical reaction in response to an electrical potential applied thereto, or becomes permeable or breached mechanically in response to a force applied thereto.

21. A device as claimed in any of claims 13 to 20 in which the release control means comprises an element mounted on the substrate or the lid means, and which controls fluid flow in a fluidic pathway between a reservoir containing a substance to be released and said one or more wells.

22. A device as claimed in any preceding claim further comprising a temperature sensor and temperature control means.

23. A device as claimed in any preceding claim wherein the device further comprises one or more fluidic channel(s) open to the or each well.

24. A device as claimed in any preceding claim further comprising a memory system.

25. Apparatus including a device as claimed in any preceding claim and a transport module adapted and arranged to control the operation of said device in transit.

26. Apparatus as claimed in claim 25 in which the transport module includes a thermally insulative housing.

27. Apparatus as claimed in claim 25 or claim 26 in which the transport module includes temperature control means for controlling the temperature of the contents of the one or more wells.

28. Apparatus as claimed in any of claims 25 to 27 wherein the transport module further comprises a heat sink.

29. Apparatus as claimed in claim 28 wherein the heat sink is maintained at a temperature below stabilising temperature of the inside of the apparatus and/or device.

30. Apparatus as claimed in either claim 28 or 29 wherein the heat sink comprises a cold body, comprising a material or assembly which may be cooled before introduction into the apparatus.

31. Apparatus as claimed in any of claims 28 to 30 wherein the heat sink or cold body comprises a phase change or eutectic material, for example a gel, which is adapted to absorb or release latent heat at a temperature below that at which the device is desired to be held.

32. Apparatus as claimed in any of claims 25 to 31 in which the transport module includes a source of gas, and means for controlling the gaseous environment adjacent the one or more wells by gas flow or diffusion.

33. Apparatus as claimed in any of claims 25 to 32 in which the transport module includes means for altering the composition of a liquid medium in the one or more wells with time.

34. Apparatus as claimed in any of claims 25 to 33 in which the transport module includes control means to control the timing and/or rate of release of the substance into the well or medium.

35. Apparatus as claimed in any one of claims 25 to 34 in which the device is provided with a temperature sensor for sensing the temperature of the contents of the wells.

36. Apparatus as claimed in any one of claims 25 to 35 in which the device is provided with a plurality of different sensors for sensing the conditions in the device, and the transport module is provided with means for recording the conditions as a function of time.

37. Apparatus as claimed in any one of claims 25 to 36 wherein the device and/or transport module provides means for prompting or prevention of intervention by a user in respect of whole device objects in all or in just some of the wells.

38. A transport module for use in apparatus as claimed in any one of claims 25 to 37.

39. A transport module as claimed in claim 38 including a communications interface for attachment to wireless communications apparatus to transmit data related to the transport module and/or an associated device.

40. A transport module as claimed in either claim 38 or 39 including a communications interface for attachment to wireless communications apparatus to receive control signals from a remote location.

41. An apparatus for transporting a payload at a controlled temperature, the apparatus comprising an outer housing, an outer thermally insulating region, an inner thermally insulating region, and a heat sinking region located between the inner and outer thermally insulating regions, the inner thermally insulating region defining a cavity for receiving a payload.

42. An apparatus as claimed in claim 41 in which the outer housing comprises the outer thermally insulating region.

43. An apparatus as claimed in claim 41 or 42 in which the outer thermally insulating region comprises one or more thermally insulating elements.

44. An apparatus as claimed in any one of claims 41 to 43 in which the inner thermally insulating region comprises one or more thermally insulating elements.

45. An apparatus as claimed in any one of claims 41 to 44 in which the inner thermally insulating region comprises one or more vacuum insulating panels.

46. An apparatus as claimed in any one of claims 41 to 45 in which the outer thermally insulating region comprises one or more vacuum insulating panels.

47. An apparatus as claimed in any one of claims 41 to 46 in which the heat sinking region comprises one or more heat absorbing elements.

48. An apparatus as claimed in claim 47 in which the one or more heat absorbing elements comprise a phase change material.

49. An apparatus as claimed in any one of claims 41 to 48 in which the heat sinking region comprises one or more removable bodies which may be cooled.

50. An apparatus as claimed in any one of claims 41 to 49 further comprising heating means for heating a payload in use.

51. An apparatus as claimed in any one of claims 41 to 50 and a removable payload unit, said payload unit including a receiving region for a payload and means for heating the payload in use.

52. An apparatus as claimed in claim 51, the payload unit further including the inner insulation region.

53. An apparatus as claimed in claim 52, the payload unit further including the heat sinking region.

54. An apparatus as claimed in any of claims 50 to 53 in which the heating means comprises an electric heater.
